(19) United States
(12) Patent Application Publication
(10) Pub. No.: US 2023/0235408 A1
(43) Pub. Date: Jul. 27, 2023

(54) METHODS FOR PREDICTING THE RISK OF RECURRENCE AND/OR DEATH OF PATIENTS SUFFERING FROM A SOLID CANCER AFTER PREOPERATIVE ADJUVANT THERAPIES

(71) Applicants: INSERM (INSTITUT NATIONAL DE LA SANTÉ ET DE LA RECHERCHE MÉDICALE), Paris (FR); ASSISTANCE PUBLIQUE-HÔPITAUX DE PARIS (APHP), Paris (FR); SORBONNE UNIVERSITÉ, Paris (FR); UNIVERSITÉ DE PARIS, Paris (FR)

(72) Inventors: Franck PAGES, Paris cedex 15 (FR); Jérôme GALON, Paris (FR); Guy ZEITOUN, Paris cedex 15 (FR); Amos KIRILOVSKY, Paris cedex 06 (FR)

(21) Appl. No.: 18/003,535

(22) PCT Filed: Jun. 28, 2021
(86) PCT No.: PCT/EP2021/067738

§ 371 Date: Dec. 28, 2022

(30) Foreign Application Priority Data
Jun. 30, 2020 (EP) 20305730.2

(51) Int. Cl. C12Q 1/6886; G01N 33/574; G01N 33/569

(52) U.S. Cl. CPC C12Q 1/6886; G01N 33/57419; G01N 33/56972; G01N 2800/52; C12Q 1/6869

(57) ABSTRACT

The inventors assessed in locally advanced rectal cancer whether a diagnostic biopsy-adapted Immunoscore (ISg) could predict response to neoadjuvant treatment (nT) and better define patients eligible to an organ preservation strategy (“Watch-and-Wait”). The inventors showed that IS_{B} was an independent parameter, more informative than pre- (P<0.001) and post-nT (P<0.05) imaging to predict disease-free survival. IS_{B} combined with imaging post-nT discriminated very good responders that could benefit from organ preservation strategy. Accordingly, the present invention relates to methods for predicting the recurrence and/or death of patients suffering from a solid cancer after preoperative adjuvant therapies.

### FIELD OF THE INVENTION

The present invention is in the field of medicine, in particular oncology and immunology.

### BACKGROUND OF THE INVENTION

Colorectal cancer is the third most common cancer in the world with an increasing incidence especially in younger adults (1). In locally advanced rectal cancer (LARC) neoadjuvant chemoradiotherapy (nCRT) followed by radical surgery is recommended by international guidelines (2,3). Tumor recurrence and patient's survival are strongly influenced by the quality of response to neoadjuvant treatment (nT) (4-6). Recent advances in the management of LARC patients have shown that it could be conceivable to avoid amputation of the rectum (preservative strategy; eg. Watch and Wait) in patients with clinical and imaging features compatible with a complete response to nT (7,8). These patients experience acceptable outcomes, however, about 25% of them develop early tumor regrowth (9). There are currently no molecular markers to predict responses to nCRT, and guide treatment decision (3), such as optimization or modification of nT in non-responding patients and better selection of patients eligible to preservative strategy.

Ionizing radiation has the capacity to prime/reinforce an adaptive T-cell mediated immune response, which acts in the mechanisms of local tumor regression and of distant tumor inhibition and rejection (i.e. the abscopal effect) (10-12). This suggests that the quality and intensity of the natural immune reaction at tumor site before nT could influence the magnitude of response to nT and provide a predictive marker of response. Natural immune reaction at tumor site has been further associated with a favorable prognosis in various cancers (13), including colorectal cancer, treated by surgery alone (14,15). Recent advances in digital pathology and image analysis have allowed a translation of immune assessment into a clinically relevant application (16). Using these technologies, the first standardized immune-based assay for colon cancer called “Immunoscore” (IS; i.e. the combination of CD3+ and CD8+ T cell densities in the tumor and its invasive margin) has been developed. Its robustness and prognostic performance in stage I-III colon cancer has been consolidated through an international validation study (17). Thus, IS provides a reliable evaluation of the natural immune reaction at tumor site.

Preliminary studies in rectal cancer have suggested that the natural immune reaction of tumors could be evaluated on biopsies (18-20), the only sample material available before treatment. A derivation of the Immunoscore performed in initial biopsies (IS_{B}) before nT have the benefit of evaluating the quality of the initial immune response in the tumor and its potential influence on both the degree of response to nT and clinical outcome.

### SUMMARY OF THE INVENTION

The present invention is defined by the claims. In particular, the present invention relates to methods for predicting the risk of recurrence and/or death of patients suffering from a solid cancer after preoperative adjuvant therapies.

### DETAILED DESCRIPTION OF THE INVENTION

The inventors showed that biopsy-adapted Immunoscore (IS_{B}) performed on cancer biopsy samples and combined with imaging post-neoadjuvant radiochemotherapy (nCRT) discriminate the group of patients with a complete histological response to nCRT (no residual tumor) that should benefit from less invasive therapeutic strategies (ie. Watch-and-Wait or minimally invasive surgery), avoiding a disabling and useless rectal amputation surgery.

### Definitions

As used herein, the term “tumor” refers to all neoplastic cell growth and proliferation, whether malignant or benign, and all pre-cancerous and cancerous cells and tissues.

As used herein, the term “cancer” refer to or describe the physiological condition in mammals that is typically characterized by unregulated cell growth. The term “cancer” as used herein includes carcinomas, (e.g., carcinoma in situ, invasive carcinoma, metastatic carcinoma) and pre-malignant conditions, neomorphic changes independent of their histological origin. The term “cancer” is not limited to any stage, grade, histomorphological feature, invasiveness, aggressiveness or malignancy of an affected tissue or cell aggregation. In particular stage 0 cancer, stage I cancer, stage II cancer, stage III cancer, stage IV cancer, grade I cancer, grade II cancer, grade III cancer, malignant cancer and primary carcinomas are included.

As used herein, the term “primary cancer” refers to the original, or first, tumor in the body. Cancer cells from a primary tumor may spread to other parts of the body and form new, or secondary, tumors (i.e. metastasis).

As used herein, the term “locally advanced cancer” refers to cancer that has spread from where it started in the organ tissue to nearby tissue or lymph nodes, but not to other parts of the body.

As used herein, the term “metastatic cancer” refers to a cancer that has spread from the place where it first started to another place in the body and specifically to the lymph node.

As used herein, the term “colorectal cancer” includes the well-accepted medical definition that defines colorectal cancer as a medical condition characterized by cancer of cells of the intestinal tract below the small intestine (i.e., the large intestine (colon), including the cecum, ascending colon, transverse colon, descending colon, sigmoid colon, and rectum). Additionally, as used herein, the term “colorectal cancer” also further includes medical conditions, which are characterized by cancer of cells of the duodenum and small intestine (jejunum and ileum). As used herein, the term “a microsatellite unstable colorectal cancer” refers to a colorectal cancer characterized by microsatellite instability.

As used herein, the term “microsatellite instability” or “MSI” has its general meaning and is defined as the accumulation of insertion-deletion mutations at short repetitive DNA sequences (or ‘microsatellites’) is a characteristic feature of cancer cells with DNA mismatch repair (MMR) deficiency. Inactivation of any of several MMR genes, including MLH1, MSH2, MSH6 and PMS2, can result in MSI. Originally, MSI was shown to correlate with germline defects in MMR genes in patients with Lynch syndrome (LS), where >90% of colorectal cancer (CRC) patients exhibit MSI. It was later recognized that MSI also occurs in ^{˜}12% of sporadic CRCs occurring in patients that lack germline MMR mutations, and MSI in these patients is due to promoter methylation-induced silencing of the MLH1 gene expression. Determination of MSI status in CRC involves routine methods well known in the art.

As used herein, the term “recurrence” refers to a return of the cancer, either locally (e.g., where it used to be before therapy) or distally (e.g., metastasis).

As used herein, the term “risk” in the context of the present invention, relates to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion, thus diagnosing and defining the risk spectrum of a category of subjects defined as being at risk of conversion.

As used herein, the term “time to recurrence” or “TTR” is used herein to refer to time in years to first recurrence censoring for second primary cancer as a first event or death without evidence of recurrence.

As used herein, the term “survival time” includes “progression-free survival”, “death-free survival” and “overall survival”.

As used herein, the term “progression-free survival” or “PFS” in the context of the invention refers to the length of time during and after treatment during which, according to the assessment of the treating physician or investigator, the patient's disease does not become worse, i.e., does not progress. As the skilled person will appreciate, a patient's progression-free survival is improved or enhanced if the patient experiences a longer length of time during which the disease does not progress as compared to the average or mean progression free survival time of a control group of similarly situated patients.

As used herein, the term “disease-free survival” or “DFS” has its general meaning in the art and is defined as the time from randomization to recurrence of tumor or death, and it is typically used in the adjuvant treatment setting. The term is also known as “relapse-free survival”.

As used herein, the term “overall survival” or “OS” in the context of the invention refers to the average survival of the patient within a patient group. As the skilled person will appreciate, a patient's overall survival is improved or enhanced, if the patient belongs to a subgroup of patients that has a statistically significant longer mean survival time as compared to another subgroup of patients. Improved overall survival may be evident in one or more subgroups of patients but not apparent when the patient population is analyzed as a whole.

As used herein, the expression “short survival time” indicates that the subject will have a survival time that will be lower than the median (or mean) observed in the general population of subjects. When the subject will have a short survival time, it is meant that the subject will have a “poor prognosis”. Inversely, the expression “long survival time” indicates that the subject will have a survival time that will be higher than the median (or mean) observed in the general population of subjects. When the subject will have a long survival time, it is meant that the subject will have a “good prognosis”.

As used herein, the term “therapy” refers to a timely sequential or simultaneous administration of anti-tumor, and/or anti vascular, and/or anti stroma, and/or immune stimulating or suppressive, and/or blood cell proliferative agents, and/or radiation therapy, and/or hyperthermia, and/or hypothermia for cancer therapy. The administration of these can be performed in an adjuvant and/or neoadjuvant mode. The composition of such protocol may vary in the dose of each of the single agents, timeframe of application and frequency of administration within a defined therapy window. Currently various combinations of various drugs and/or physical methods, and various schedules are under investigation.

As used herein, the term “preoperative adjuvant therapy” or “neoadjuvant therapy” refers to a preoperative therapy regimen consisting of a panel of therapies that can include e.g. chemotherapy, radiotherapy, targeted therapy, hormone therapy and/or immunotherapy which is aimed to shrink the primary tumor, thereby rendering local therapy (e.g. surgery) less destructive or more effective, or enabling conserving surgery or enabling organ preservation.

As used herein, the term “chemotherapy” has its general meaning in the art and refers to the treatment that consists in administering to the patient a chemotherapeutic agent.

As used herein, the term “chemotherapeutic agent” refers to a chemical compound that is (i.e., drug) or becomes (i.e., prodrug), for example, selectively destructive or selectively toxic to malignant cells and tissues.

As used herein, the term “immunotherapy” has its general meaning in the art and refers to the treatment that consists in administering an immunogenic agent i.e. an agent capable of inducing, enhancing, suppressing or otherwise modifying an immune response. In some embodiments, the immunotherapy consists in administering the patient with at least one immune checkpoint inhibitor.

As used herein, the term “immune checkpoint inhibitor” has its general meaning in the art and refers to any compound inhibiting the function of an immune inhibitory checkpoint protein. As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA. Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules. Examples of immune checkpoint inhibitor includes PD-1 antagonist, PD-L1 antagonist, PD-L2 antagonist CTLA-4 antagonist, VISTA antagonist, TIM-3 antagonist, LAG-3 antagonist, IDO antagonist, KIR2D antagonist, A2AR antagonist, B7-H3 antagonist, B7-H4 antagonist, and BTLA antagonist.

As used herein, the term “radiotherapy” has its general meaning in the art and refers a therapy with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

As used herein the term “hypofractionated radiation therapy” has its general meaning in the art and refers to radiation therapy in which the total dose of radiation is divided into large doses and treatments are given less than once a day.

In some embodiments, the term “contact radiotherapy” has its general meaning in the art and refers to radiotherapy for which the irradiation (e.g. low energy X-rays treatment) is carried out with a device that comprises an applicator that is intended to be in contact with the tissue to be treated. Contact radiotherapy. Typically, contact radiotherapy involves the Papillon thechnique (Sun Myint A, Stewart A, Mills J, et al. Treatment: the role of contact X-ray brachytherapy (Papillon) in the management of early rectal cancer. Colorectal Dis. 2019; 21 Suppl 1:45-52).

As used herein, the term “targeted therapy” refers to a therapy targeting a particular class of proteins involved in tumor development or oncogenic signaling. For example, tyrosine kinase inhibitors against vascular endothelial growth factor have been used in treating cancers.

As used herein, the term “hormone therapy” or “hormonal therapy” refers to the therapy that consists in reducing, blocking, or inhibiting the effects of hormones that can promote the growth of cancer. As used herein, the term “hormone therapy agent” refers to anti-androgens (including steroidal anti-androgens and non-steroidal anti-androgens), estrogens, luteinizing hormone-releasing hormone (LIR) agonists, and LHIRH antagonists, as well as, hormonal ablation therapy.

As used herein, the term “surgery” applies to surgical methods undertaken for removal of cancerous tissue, including mastectomy, lumpectomy, lymph node removal, sentinel lymph node dissection. In particular, the term “radical surgery” also called “radical dissection”, is surgery that is more extensive than “conservative surgery” and is intended to remove both a tumor and any metastases thereof, for treatment purposes.

As used herein, the term “adjuvant therapy” refers to any type of therapy of cancer given as additional treatment, usually after surgical resection of the primary tumor, in a patient affected with a cancer that is at risk of metastasizing and/or likely to recur. The aim of such an adjuvant treatment is to improve the prognosis. Adjuvant therapies comprise radiotherapy and therapy, preferably systemic therapy, such as hormone therapy, chemotherapy, immunotherapy and monoclonal antibody therapy.

As used herein, the term “responsive” refers to a patient that achieves a response, i.e. a patient where the cancer is eradicated, reduced or improved. The patient is thus qualified as a “responder”. According to the invention, the responders have an objective response and therefore the term does not encompass patients having a stabilized cancer such that the disease is not progressing after the preoperative adjuvant therapy. A “non-responder” or “refractory” patient includes patients for whom the cancer does not show reduction or improvement after the preoperative adjuvant therapy. According to the invention, the term “non-responder” also includes patients having a stabilized cancer.

As used herein, the term “clinical response” refers to the response to the preoperative adjuvant therapy that is assessed by any clinical method well known in the art. For instance, the clinical response may be assessed where the size of a tumor after systemic intervention can be compared to the initial size and dimensions as measured by imaging and/or quantification of a biomarker. Thus in particular, the clinical response refers to a change in tumor mass and/or volume after initiation of the preoperative adjuvant therapy and/or prolongation of time to distant metastasis or time to death following the preoperative adjuvant therapy. Response may be recorded in a quantitative fashion or in a qualitative fashion like “no change” (NC), “partial remission” (PR), “complete remission” (CR) or other qualitative criteria. Assessment of the clinical response may be done early after the onset of the preoperative adjuvant therapy e.g. after a few hours, days, weeks or preferably after a few months. A typical endpoint for response assessment is upon termination of the preoperative adjuvant therapy. This is typically three month after initiation of the preoperative adjuvant therapy.

As used herein, the term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

As used herein, the term “immune response” includes both innate and adaptive immune response that results in selective damage to, destruction of, or elimination from the human body of tumor cells. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

As used herein, the term “biomarker” has its general meaning in the art and refers to any molecule that is detectable in a sample. Such molecules may include peptides/proteins or nucleic acids and derivatives thereof.

As used herein, the term “immune marker” consists of any detectable, measurable and quantifiable parameter that is indicative of the status of the immune response of the cancer patient against the tumor. In the present specification, the name of each of the various immune markers of interest refers to the internationally recognised name of the corresponding gene, as found in internationally recognised gene sequences and protein sequences databases, including in the database from the HUGO Gene Nomenclature Committee. In the present specification, the name of each of the various immune markers of interest may also refer to the internationally recognised name of the corresponding gene, as found in the internationally recognised gene sequences and protein sequences database Genbank. Through these internationally recognised sequence databases, the nucleic acid and the amino acid sequences corresponding to each of the immune marker of interest described herein may be retrieved by the one skilled in the art. Immune markers include the presence of, or the number or density of, cells from the immune system at the tumor site. Immune markers also include the presence of, or the amount of proteins specifically produced by cells from the immune system at the tumor site. Immune markers also include the presence of, or the amount of, any biological material that is indicative of the expression level of genes related to the raising of a specific immune response of the host, at the tumor site. Thus, immune markers include the presence of, or the amount of, messenger RNA (mRNA) transcribed from genomic DNA encoding proteins which are specifically produced by cells from the immune system, at the tumor site. Immune markers thus include surface antigens that are specifically expressed by cells from the immune system, including by B lymphocytes, T lymphocytes, monocytes/macrophages dendritic cells, NK cells, NKT cells, and NK-DC cells, that are recruited within the tumor tissue, or alternatively mRNA encoding for said surface antigens. Illustratively, surface antigens of interest used as immune markers include CD3, CD4, CD8 and CD45RO that are expressed by T cells or T cell subsets. For example, if the expression of the CD3 antigen, or the expression of the mRNA thereof, is used as an immune marker, the quantification of this immune marker, at step a) of the method according to the invention, is indicative of the level of the adaptive immune response of the patient involving all T lymphocytes and NKT cells. For instance, if the expression of the CD8 antigen, or the expression of the mRNA thereof, is used as an immune marker, the quantification of this immune marker, at step a) of the method according to the invention, is indicative of the level of the adaptive immune response of the patient involving cytotoxic T lymphocytes. For example, if the expression of the CD45RO antigen, or the expression of the mRNA thereof, is used as an immune marker, the quantification of this immune marker, at step a) of the method according to the invention, is indicative of the level of the adaptive immune response of the patient involving memory T lymphocytes or memory effector T lymphocytes. Yet illustratively, proteins used as immune markers also include cytolytic proteins specifically produced by cells from the immune system, like perforin, granulysin and also granzyme-B.

As used herein the expression “gene representative of the adaptive immune response” refers to any gene that is expressed by a cell that is an actor of the adaptive immune response in the tumor or that contributes to the settlement of the adaptive immune response in the tumor. The adaptive immune response, also called “acquired immune response”, comprises antigen-dependent stimulation of T cell subtypes, B cell activation and antibody production. For example cells of the adaptive immune response include but are not limited to cytotoxic T cells, T memory T cells, Th1 and Th2 cells, activated macrophages and activated dendritic cells, NK cells and NKT cells.

As used herein the expression “gene representative of the immunosuppressive response” refers to any gene that is expressed by a cell that is an actor of the immunosuppressive response in the tumor or that contributes to the settlement of the immunosuppressive response in the tumor. For example, the immunosuppressive response comprises
co-inhibition of antigen-dependent stimulation of T cell subtypes: genes CD276, CTLA4, PDCD1, CD274, TIM-3 or VTCN1 (B7H4),
inactivation of macrophages and dendritic cells and inactivation of NK cells: genes TSLP, CD1A, or VEGFA
expression of cancer stem cell marker, differentiation and/or oncogenesis: PROM1, IHH.
expression of immunosuppressive proteins produced in the tumour environment: genes PF4, REN, VEGFA.

For example cells of the immunosuppressive response include immature dendritic cells (CD1A), regulatory T cells (Treg cells) and Th17 cells expressing IL17A gene.

As used herein, the term “sample” refers to any sample obtained from the subject for the purpose of performing the method of the present invention. In some embodiments, the sample is a bodily fluid (e.g. a blood sample), a population of cells, or a tissue. Examples of such bodily fluids include, blood, saliva, tears, semen, vaginal discharge, pus, mucous, urine and feces.

As used herein, the term “blood sample” refers to a whole blood sample, serum sample and plasma sample. A blood sample may be obtained by methods known in the art including venipuncture or a finger stick. Serum and plasma samples may be obtained by centrifugation methods known in the art. The sample may be diluted with a suitable buffer before conducting the assay.

As used herein, the term “tumor biopsy sample” refers to a tumor sample that result from a biopsy performed in the primary tumour of the patient or performed in metastatic sample distant from the primary tumor of the patient. For example an endoscopical biopsy performed in the bowel of the patient affected by a colorectal cancer.

As used herein, the term “parameter” refers to any characteristic assessed when carrying out the method according to the invention. As used herein, the term “parameter value” refers to a value (a number for instance) associated to a parameter.

As used herein, the term “score” refers to a numeric value that is derived by combining one or more parameter in a mathematic algorithm or formula. Combining the parameters can be accomplished for example by multiplying each expression level with a defined and specified coefficient and summing up such products to yield a score. The score may be determined by a scoring system that can be a continuous scoring system or a non-continuous scoring system.

As used herein, the term “scoring system” refers to any method in which the application of an agreed numerical scale is used as a means of estimating the degree of a response (i.e. the immune response or the clinical response).

As used herein, the term “automated scoring system” means that the scoring system is in part or entirely controlled and carried out by machine (e.g. a computer) and, hereby limiting the human input.

As used herein, the term “continuous scoring system” refers to a scoring system into which one or more variables that is input are continuous. The term “continuous” indicates that the variable can take on any value between its minimum value and its maximum value. In some embodiments, the value input into the continuous scoring system is the actual magnitude of the variable. In some embodiments, the value input into the continuous scoring system is the absolute value of the variable. In some embodiments, the value input into the continuous scoring system is a normalized value of the variable. Conversely, the term “non-continuous scoring system” or “binary scoring system” each variable is assigned to a pre-determined “bin” (for example, “high”, “intermediate” or “low”). For example, if the variable being assessed is a density of CD3+ T-cells, in a continuous scoring system, the value input into the function is the density of CD3+ T-cells and in a non-continuous scoring system, the density value is first analyzed to determine whether it falls into a “high density”, a “medium density” or a “low density”. Thus, consider two samples, a first having a density of 1000 CD3+ cells/mm² and a second having a density of 500 CD3+ cells/mm², the values input into a continuous scoring system would be 500 and 700, respectively and the values input into a non-continuous scoring system would depend on the bin in which they fall. If the “high bin” encompasses both 500 and 1000 cells/mm2, then a value of 1 would be input into the non-continuous scoring system for each sample. If the cut-off value between “high” and “low” bins fell somewhere between 500 and 1000 cells/mm², then a value of “high” would be input into the non-continuous scoring system for the first sample, and a value of “low” would be input into the non-continuous scoring system for the second sample. A useful way of determining such cut-off value is to construct a receiver-operator curve (ROC curve) on the basis of all conceivable cut-off values, determine the single point on the ROC curve with the closest proximity to the upper left corner (0/1) in the ROC plot. Obviously, most of the time cut-off values will be determined by less formalized procedures by choosing the combination of sensitivity and specificity determined by such cut-off value providing the most beneficial medical information to the problem investigated. Please note that these values are intended to illustrate the difference between a continuous scoring system and a non-continuous scoring system, and should not be construed as in any way limiting the scope of the disclosure unless recited in a claim.

As used herein, the term “TNM classification” has its general meaning in the art and refers to the classification published by the Union for International Cancer Control (UICC). The UICC TNM classification is the internationally accepted standard for cancer staging. The UICC TNM Classification is an anatomically based system that records the primary and regional nodal extent of the tumor and the absence or presence of metastases. Each individual aspect of TNM is termed as a category. T category describes the extent of the primary tumour Ta, T0, Tis, T1, T2, T3, T4, Tx N-category. N category describes the absence or presence and extent of regional lymph node metastasis N0, N1, N2, N3, Nx M-category. M category describes the absence or presence of distant metastasis M0, M1, Mx. Cancer in situ is categorized stage 0; often tumors localized to the organ of origin are staged as I or II depending on the extent, locally extensive spread, to regional nodes are staged as III, and those with distant metastasis staged as stage IV. To indicate that the clinical or pathologic classification has been determined after preoperative adjuvant therapy, the TNM classification includes a prefix “y,” with yc indicating the clinical classification and yp the pathologic classification. In those cases in which classification is performed during or after initial multimodality therapy, the cTNM or pTNM category is identified by a “y” prefix. The ycTNM or ypTNM thus categorizes the extent of tumor that actually is present at the time of each respective examination. The following illustrates the use of the “y” prefix. A patient presents with a rectal tumor. Preoperative imaging shows that the tumor extends into the perirectal fat. There is 1 enlarged perirectal lymph node and no evidence of distant metastases. The patient receives preoperative chemoradiation. Before surgery, there is no evidence of the tumor on clinical and radiologic examination, and a clinical complete response has been achieved. Surgery is performed and the pathology report reveals residual tumor invading into the submucosa. There is no evidence of tumor in 16 lymph nodes, but 1 lymph node contains a mucin lake. For this patient, the TNM classification is
before any treatment: cT3N1M0
after neoadjuvant therapy: ycT0N0M0
after surgery: ypT1N0M0

As used herein, the term “immunoscore” refers to the combination of CD3+ and CD8+ T cell densities determined in the tumor biopsy sample obtained from the patient as described in the EXAMPLE. Immunoscore® is a registered trademark from INSERM (Institut National De La Sante Et De La Recherche Medicale)—France). In particular, Inserm is the owner of the trademark “IMMUNOSCORE” duly protected in United States of America through the International Registration no. 1146519 in classes 01, 05, 09, 10, 42 and 44.

As used herein, the term “percentile” has its general meaning in the art and refers to a measure used in statistics indicating the value below which a given percentage of observations in a group of observations falls. For example, the 20th percentile is the value (or score) below which 20% of the observations may be found. Equivalently, 80% of the observations are found above the 20th percentile. The term percentile and the related term percentile rank are often used in the reporting of scores from norm-referenced tests. For example, if a score is at the 86th percentile, where 86 is the percentile rank, it is equal to the value below which 86% of the observations may be found (carefully contrast with in the 86th percentile, which means the score is at or below the value below which 86% of the observations may be found—every score is in the 100th percentile). The 25th percentile is also known as the first quartile (Q1), the 50th percentile as the median or second quartile (Q2), and the 75th percentile as the third quartile (Q3). In general, percentiles and quartiles are specific types of quantiles.

As used herein, the term “arithmetic mean value” has its general meaning in the art and refers to the quantity obtained by summing two or more numbers or variables and then dividing by the number of numbers or variables.

As used herein, the term “median value” has its general meaning in the art and refers to the value that separated the higher half from the lower half of a data sample, a population or a probability distribution. For a data set, it may be thought of as “the middle” value.

As used herein, the term “combination” or “combining” is defined as a possible selection of a certain number of parameters and the arrangement of these parameters into specified groups using a mathematical formula or algorithm.

As used herein, the term “circulating-tumor DNA” or “ctDNA” has its general meaning in the art and refers to DNA that comes from cancerous cells and that is found in the bloodstream.

As used herein, the term “radiography” refers to a recording technique that makes use of penetrating radiation, which includes highly energetic radiation such as X-rays, γ-rays, β-rays and fast electrons.

As used herein, the term “ultrasound imaging” refer to a medical imaging modality that employs sound waves (e.g., with a frequency greater than or equal to 20,000 Hz) to produce real-time images (or “sonograms”) of an internal structure of a patient's body. In some embodiments, the internal structure of the patient's body can be opaque to light. The terms “ultrasound imaging”, “sonography”, and “acoustic imaging” can be used interchangeably herein. As used herein, the term “three-dimensional” ultrasound can refer to an ultrasound image that is valid and accurate in three dimensions. An example of a three-dimensional ultrasound modality can include contrast-enhanced ultrasound (“CEUS”) imaging.

As used herein, the term “magnetic resonance imaging” or “MRI”, also known as magnetic resonance tomography (MRT), relates to a medical imaging technique most commonly used in radiology to visualize the structure and function of the body. It provides detailed images of the body in any plane. MRI uses non ionizing radiation, but uses a powerful magnetic field to align the nuclear magnetization of (usually) hydrogen atoms in water in the body. Radio frequency fields are used to systematically alter the alignment of this magnetization, causing the hydrogen nuclei to produce a rotating magnetic field detectable by the scanner. This signal can be manipulated by additional magnetic fields to build up enough information to construct an image of the body. When a subject lies in a scanner, the hydrogen nuclei (i.e., protons) found in abundance in an animal body in water molecules, align with the strong main magnetic field. A second electromagnetic field that oscillates at radio frequencies and is perpendicular to the main field, is then pulsed to push a proportion of the protons out of alignment with the main field. These protons then drift back into alignment with the main field, emitting a detectable radio frequency signal as they do so. Since protons in different tissues of the body (e.g., fat versus muscle) realign at different speeds, the different structures of the body can be revealed.

As used herein, the term “scintigraphy” refers to a recording technique which a two-dimensional image of a body having a radiation source is obtained through the use of radioisotopes. A radioactive chemical is injected into the patient which then concentrates in the target cells or organ of interest. By placing a camera that senses radioactivity over the body, an image of the target cells or organ of interest can be created. The particles can be detected by suitable devices such as gamma cameras, positron emission tomography (PET) machines, single photon emission computed tomography (SPECT) machines and the like.

As used herein, the term “positron emission tomography imaging” or “PET imaging” is used herein to refer to the use of PET to capture images, particularly of a living body. It is to be understood that the term includes both dynamic and still images received as a result of the technique. “Positron emission tomography” (PET) is a nuclear medicine imaging technique which produces a three-dimensional image or picture of functional processes in the body. The PET scanner detects pairs of gamma rays emitted indirectly by a positron-emitting radionuclide (tracer), which is introduced into the body on a biologically active molecule. Images of tracer concentration in 3-dimensional space within the body are then reconstructed by computer analysis.

As used herein, the term “computed tomography” or “CT” refers to a medical imaging method employing tomography where digital geometry processing is used to generate a three-dimensional image of the internals of an object from a large series of two-dimensional X-ray images taken around a single axis of rotation. The term as used herein is non-exclusive, and includes CT-based methods and combination methods, such as PET/CT.

As used herein, the term “algorithm” is any mathematical equation, algorithmic, analytical or programmed process, or statistical technique that takes one or more continuous parameters and calculates an output value, sometimes referred to as an “index” or “index value.”

As used herein, the term “digital pathology” is a sub-field of pathology that focuses on data management based on information generated from digitized specimen slides. It will be understood that such images will have features in the images representing tissue features, such as shape and color, and texture. These features can be extracted in quantitative form through the use of computer-based technology.

### Method of the Present Invention

The present invention relates to a method of predicting the risk of recurrence and/or death of a patient suffering from a solid cancer after preoperative adjuvant therapy comprising the step of assessing at least two parameters, wherein the first parameter is the immune response determined before the preoperative adjuvant therapy and the second parameter is the clinical response determined after the preoperative adjuvant therapy and wherein the combination of said parameters indicates the risk of recurrence and/or death.

In some embodiments, the method of the present invention is particularly suitable for predicting time to recurrence.

In some embodiments, the method of the present invention is particularly suitable for predicting the survival time of the patient. In particular, the method of the present invention is particularly suitable for predicting the duration of the overall survival (OS), progression-free survival (PFS) and/or the disease-free survival (DFS) of the cancer patient. More particularly, the method of the present invention is particularly suitable for predicting the disease-free survival.

Cancers:

Typically the patient subjected to the above method may suffer from a solid cancer selected from the group consisting adrenal cortical cancer, anal cancer, bile duct cancer (e.g. periphilar cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, multiple myeloma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ, gynecomastia), cervical cancer, colorectal cancer, endometrial cancer (e.g. endometrial adenocarcinoma, adenocanthoma, papillary serous adnocarcinoma, clear cell), esophagus cancer, gallbladder cancer (mucinous adenocarcinoma, small cell carcinoma), gastrointestinal carcinoid tumors (e.g. choriocarcinoma, chorioadenoma destruens), Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma), vaginal cancer, vulvar cancer, and uterine cancer (e.g. uterine leiomyosarcoma).

In some embodiments, the patient suffers from a primary cancer. In some embodiments, the patient suffers from a locally advanced cancer. In some embodiments, the patient suffers from a stage II TNM cancer. In some embodiments, the patient suffers from a stage III TNM cancer.

In some embodiments, the patient suffers from a metastatic cancer. In some embodiments, the patient suffers from a stage IV TNM cancer.

In some embodiments, the patient suffers from an esophageal cancer, a rectal cancer, a colon cancer, a breast cancer, a lung cancer, a prostate cancer, a head and neck cancer or a liver cancer. In some embodiments, the patient suffers from a colorectal cancer and more particularly from a rectal cancer. In some embodiments, the patient suffers from a locally advanced rectal cancer.

Preoperative Adjuvant Therapies:

In some embodiments, the preoperative adjuvant therapy consists of a radiotherapy, a chemotherapy, a targeted therapy, a hormone therapy, an immunotherapy or a combination thereof. In some embodiments, the preoperative adjuvant therapy consists of a combination of radiotherapy and chemotherapy.

Non limiting examples of targeted that can be used in the preoperative adjuvant targeted therapy are selected from inhibitors of HER1/EGFR (EGFRvIII, phosphorylated (p-) EGFR, EGFR:Shc, ubiquitinated (u-) EGFR, p-EGFRvIII); ErbB2 (p-ErbB2, p95HER2 (truncated ErbB2), p-p95HER2, ErbB2:Shc, ErbB2:PI3K, ErbB2:EGFR, ErbB2:ErbB3, ErbB2:ErbB4); ErbB3 (p-ErbB3, truncated ErbB3, ErbB3:PI3K, p-ErbB3:PI3K, ErbB3: Shc); ErbB4 (p-ErbB4, ErbB4:Shc); c-MET (p-c-MET, truncated c-MET, c-Met:HGF complex); AKT1 (p-AKT1); AKT2 (p-AKT2); AKT3 (p-AKT3); PTEN (p-PTEN); P70S6K (p-P70S6K); MEK (p-MEK); ERK1 (p-ERK1); ERK2 (p-ERK2); PDK1 (p-PDK1); PDK2 (p-PDK2); SGK3 (p-SGK3); 4E-BP1 (p-4E-BP1); PIK3R1 (p-PIK3R1); c-KIT (p-c-KIT); ER (p-ER); IGF-1R (p-IGF-1R, IGF-1R:IRS, IRS:PI3K, p-IRS, IGF-1R:PI3K); INSR (p-INSR); FLT3 (p-FLT3); HGFR1 (p-HGFR1); HGFR2 (p-HGFR2); RET (p-RET); PDGFRA (p-PDGFRA); PDGFRB (p-PDGFRB); VEGFR1 (p-VEGFR1, VEGFR1:PLCT, VEGFR1:Src); VEGFR2 (p-VEGFR2, VEGFR2:PLCT, VEGFR2:Src, VEGFR2:heparin sulphate, VEGFR2:VE-cadherin); VEGFR3 (p-VEGFR3); FGFR1 (p-FGFR1); FGFR2 (p-FGFR2); FGFR3 (p-FGFR3); FGFR4 (p-FGFR4); TIE1 (p-TIE1); TIE2 (p-TIE2); EPHA (p-EPHA); EPHB (p-EPHB); GSK-30 (p-GSK-30); NFKB (p-NFKB), IKB (p-IKB, p-P65:IKB); BAD (p-BAD, BAD:14-3-3); mTOR (p-mTOR); Rsk-1 (p-Rsk-1); Jnk (p-Jnk); P38 (p-P38); STAT1 (p-STAT1); STAT3 (p-STAT3); FAK (p-FAK); RB (p-RB); Ki67; p53 (p-p53); CREB (p-CREB); c-Jun (p-c-Jun); c-Src (p-c-Src); paxillin (p-paxillin); GRB2 (p-GRB2), Shc (p-Shc), Ras (p-Ras), GAB1 (p-GAB1), SHP2 (p-SHP2), GRB2 (p-GRB2), CRKL (p-CRKL), PLCγ (p-PLCγ), PKC (e.g., p-PKCα, p-PKCβ, p-PKCδ), adducin (p-adducin), RB 1 (p-RB 1), and PYK2 (p-PYK2).

Examples of such inhibitors include the small organic molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; GW 72016 (available from Glaxo) an oral HER2 and EGFR tyrosine kinase inhibitor; PKI-166 (available from Novartis); pan-HER inhibitors such as canertinib (CI-1033; Pharmacia); non selective HER inhibitors such as Imatinib mesylate (Gleevec™); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino)phthalimide); tyrphostines containing nitrothiophene moieties; PD-0183805 (Warner-Lamber); quinoxalines (U.S. Pat. No. 5,804,396); tryphostins (U.S. Pat. No. 5,804,396); ZD6474 (Astra Zeneca); PTK-787 (Novartis/Schering A G); pan-HER inhibitors such as CI-1033 (Pfizer); PKI 166 (Novartis); GW2016 (Glaxo SmithKline); CI-1033 (Pfizer); EKB-569 (Wyeth); Semaxinib (Sugen); ZD6474 (AstraZeneca); PTK-787 (Novartis/Schering A G); INC-1 CI 1 (Imclone); or as described in any of the following patent publications: U.S. Pat. No. 5,804,396; WO99/09016 (American Cyanimid); WO98/43960 (American Cyanamid); WO97/38983 (Warner Lambert); WO99/06378 (Warner Lambert); WO99/06396 (Warner Lambert); WO96/30347 (Pfizer, Inc); WO96/33978 (Zeneca); W096/3397 (Zeneca); and WO96/33980 (Zeneca). In some embodiments, the HER inhibitor is an EGFR inhibitor. EGFR inhibitors are well known in the art (Inhibitors of erbB-1 kinase; Expert Opinion on Therapeutic Patents December 2002, Vol. 12, No. 12, Pages 1903-1907, Susan E Kane. Cancer therapies targeted to the epidermal growth factor receptor and its family members. Expert Opinion on Therapeutic Patents February 2006, Vol. 16, No. 2, Pages 147-164. Peter Traxler Tyrosine kinase inhibitors in cancer treatment (Part II). Expert Opinion on Therapeutic Patents December 1998, Vol. 8, No. 12, Pages 1599-1625). Examples of such agents include antibodies and small organic molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a folly human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF (see WO98/50433, Abgenix); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding; and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). Examples of small organic molecules that bind to EGFR include ZD1839 or Gefitinib (IRESSA™; Astra Zeneca); CP-358774 or erlotinib (TARCEVA™; Genentech/OSI); and AG1478, AG1571 (SU 5271; Sugen); EMD-7200. In some embodiments, the HER inhibitor is a small organic molecule pan-HER inhibitor such as dacomitinib (PF-00299804). In some embodiments, the HER inhibitor is selected from the group consisting of cetuximab, panitumumab, zalutumumab, nimotuzumab, erlotinib, gefitinib, lapatinib, neratinib, canertinib, vandetanib, afatinib, TAK-285 (dual HER2 and EGFR inhibitor), ARRY334543 (dual HER2 and EGFR inhibitor), Dacomitinib (pan-ErbB inhibitor), OSI-420 (Desmethyl Erlotinib) (EGFR inhibitor), AZD8931 (EGFR, HER2 and HER3 inhibitor), AEE788 (NVP-AEE788) (EGFR, HER2 and VEGFR 1 12 inhibitor), Pelitinib (EKB-569) (pan-ErbB inhibitor), CUDC-101 (EGFR, HER2 and HDAC inhibitor), XL647 (dual HER2 and EGFR inhibitor), BMS-599626 (AC480) (dual HER2 and EGFR inhibitor), PKC412 (EGFR, PKC, cyclic AMP-dependent protein kinase and S6 kinase inhibitor), BIBX1382 (EGFR inhibitor) and AP261 13 (ALK and EGFR inhibitor). The inhibitors cetuximab, panitumumab, zalutumumab, nimotuzumab are monoclonal antibodies, erlotinib, gefitinib, lapati ib, neratinib, canertinib, vandetanib and afatinib are tyrosine kinase inhibitors.

Exemplary hormone therapy agents include, but are not limited to, cyproterone acetate, abiraterone, finasteride, flutamide, nilutamide, bicalutamide, ethylstilbestrol (DES), megestrol acetate, fosfestrol, estamustine phosphate, leuprolide, triptorelin, goserelin, histrelin, buserelin, abarelix and degarelix.

In some embodiments, the preoperative adjuvant radiotherapy is a contact radiotherapy.

Examples of chemotherapeutic agents that can be used in the preoperative adjuvant chemotherapy include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; and pharmaceutically acceptable salts, acids or derivatives of any of the above.

Examples of immune checkpoint inhibitors that can be used in the preoperative adjuvant immunotherapy include anti-CTLA4 antibodies, anti-PD1 antibodies, anti-PDL1 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies.

Examples of anti-CTLA-4 antibodies are described in U.S. Pat. Nos. 5,811,097; 5,811,097; 5,855,887; 6,051,227; 6,207,157; 6,682,736; 6,984,720; and 7,605,238. One anti-CDLA-4 antibody is tremelimumab, (ticilimumab, CP-675,206). In some embodiments, the anti-CTLA-4 antibody is ipilimumab (also known as 10D1, MDX-D010) a fully human monoclonal IgG antibody that binds to CTLA-4.

Examples of PD-1 and PD-L1 antibodies are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699. In some embodiments, the PD-1 blockers include anti-PD-L1 antibodies. In certain other embodiments the PD-1 blockers include anti-PD-1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224 is a fusion protein of B7-DC; an antibody Fc portion; BMS-936559 (MDX-1105-01) for PD-L1 (B7-H1) blockade.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211).

Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834).

Other immune-checkpoint inhibitors include TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). For example, the inhibitor can inhibit the expression or activity of TIM-3, modulate or block the TIM-3 signaling pathway and/or block the binding of TIM-3 to galectin-9. Antibodies having specificity for TIM-3 are well known in the art and typically those described in WO2011155607, WO2013006490 and WO2010117057.

In some embodiments, the immune checkpoint inhibitor is an Indoleamine 2,3-dioxygenase (IDO) inhibitor, preferably an IDO1 inhibitor. Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In some embodiments, the immune checkpoint inhibitor is an anti-TIGIT (T cell immunoglobin and ITIM domain) antibody.

Assessment of the Immune Response Before the Preoperative Adjuvant Therapy:

In some embodiments, the immune response is assessed by quantifying at least one immune marker determined in a biopsy tumor sample obtained from the patient before the preoperative adjuvant chemotherapy. Thus in some embodiments, the method comprises a step of quantifying at least one immune marker in tumor biopsy sample obtained from the patient.

In some embodiments, the tumor biopsy sample comes from the primary tumor. In some embodiments, the tumor biopsy sample comes from a metastasis.

In some embodiments, the tumor biopsy sample encompasses pieces or slices of tissue that have been removed from the tumor for further quantification of one or several immune markers, notably through histology or immunohistochemistry methods, through flow cytometry methods and through methods of gene or protein expression analysis, including genomic and proteomic analysis. The tumor biopsy sample can, of course, be subjected to a variety of well-known post-collection preparative and storage techniques (e.g., fixation, storage, freezing, etc.). The sample can be fresh, frozen, fixed (e.g., formalin fixed), or embedded (e.g., paraffin embedded). Typically the tumor biopsy sample is fixed in formalin and embedded in a rigid fixative, such as paraffin (wax) or epoxy, which is placed in a mould and later hardened to produce a block which is readily cut. Thin slices of material can be then prepared using a microtome, placed on a glass slide and submitted e.g. to immunohistochemistry (using an TIC automate such as BenchMark® XT, for obtaining stained slides). The tumour tissue sample can be used in microarrays, called as tissue microarrays (TMAs). TMA consists of paraffin blocks in which up to 1000 separate tissue cores are assembled in array fashion to allow multiplex histological analysis. This technology allows rapid visualization of molecular targets in tissue specimens at a time, either at the DNA, RNA or protein level. TMA technology is described in WO2004000992, U.S. Pat. No. 8,068,988, Olli et al 2001 Human Molecular Genetics, Tzankov et al 2005, Elsevier; Kononen et al 1198; Nature Medicine.

In said embodiments, the quantification of the immune marker is typically performed by immunohistochemistry (IHC) a described after. In said embodiments, the quantification of the marker of the immune adaptive response is typically performed by determining the expression level of at least one gene.

In some embodiments, the marker includes the presence of, or the number or density of, cells from the immune system. In some embodiments, the marker includes the presence of, or the amount of proteins specifically produced by cells from the immune system. In some embodiments, the marker includes the presence of, or the amount of, any biological material that is indicative of the level of genes related to the raising of a specific immune response of the host. Thus, in some embodiments, the marker includes the presence of, or the amount of, messenger RNA (mRNA) transcribed from genomic DNA encoding proteins which are specifically produced by cells from the immune system. In some embodiments, the marker includes surface antigens that are specifically expressed by cells from the immune system, including by B lymphocytes, T lymphocytes, monocytes/macrophages dendritic cells, NK cells, NKT cells, and NK-DC cells or alternatively mRNA encoding for said surface antigens.

When performing method of the present invention with more than one immune marker, the number of distinct immune markers that are quantified at step a) are usually of less than 100 distinct markers, and in most embodiments of less than 50 distinct markers. The number of distinct immune markers that is necessary for obtaining an accurate and reliable prognosis, using the method of the present invention, may vary notably according to the type of technique for quantification. Illustratively, high statistical significance can be found with a combination of a small number of immune markers, when the method of the present invention is performed by in situ immunohistochemical detection of protein markers of interest. Illustratively, high statistical significance was obtained with only one marker or a combination of two markers, as disclosed in the EXAMPLE. Further illustratively, high statistical significance was also found with a small number of immune markers, when the method of the present invention is performed by gene expression analysis of gene markers of interest. Without wishing to be bound by any particular theory, the inventors believe that highly statistical relevance (P value lower than 10⁻³) is reached when method of the present invention is performed by using a gene expression analysis for immune marker quantification, and by using a combination of ten distinct immune markers, and more preferably a combination of fifteen distinct immune markers, most preferably twenty distinct immune markers, or more.

Typically a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 distinct immune markers may be quantified, preferably a combination of 2, 3, 4, 5, 6, 7, 8, 9, or 10 immune markers and more preferably a combination of 2, 3, 4, 5, or 6, immune markers.

Numerous patent applications have described a large number of immune markers indicative of the status of the immune response which could be used in the methods of the invention. Typically, one can used the immune markers indicative of the status of the immune response described in WO2015007625, WO2014023706, WO2014009535, WO2013186374, WO2013107907, WO2013107900, WO2012095448, WO2012072750 and WO2007045996 (all incorporated by reference).

In some embodiments, the immune markers indicative of the status of the immune response are those described in WO2007045996.

In some embodiments, the immune markers which may be used are the cell density of cells from the immune system. In some embodiments the immune markers comprise the density of CD3+ cells, the density of CD8+ cells, the density of CD45RO+ cells, the density of GZM-B+ cells, the density of CD103+ cells and/or the density of B cells. More preferably, the immune markers comprise the density of CD3+ cells and the density of CD8+ cells, the density of CD3+ cells and the density of CD45RO+ cells, the density of CD3+ cells the density of GZM-B+ cells, the density of CD8+ cells and the density of CD45RO+ cells, the density of CD8+ cells and the density of GZM-B+ cells; the density of CD45RO+ cells and the density of GZM-B+ cells or the density of CD3+ cells and the density of CD103+ cells.

In some embodiments, the density of CD3+ cells and the density of CD8+ cells is determined in the tumor biopsy sample.

In some embodiments, the density of B-cells may also be measured (see WO2013107900 and WO2013107907). In some embodiments, the density of DC cells may also be measured (see WO2013107907).

Typically, the method disclosed in WO2013186374 may be used for quantifying the immune cells in the tumor sample.

In some embodiments, the immune markers indicative of the status of the immune response may comprise the expression level of one or more genes or corresponding proteins listed in Table 9 of WO2007045996 which are: 18s, ACE, ACTB, AGTR1, AGTR2, APC, APOA1, ARF1, AXIN1, BAX, BCL2, BCL2L1, CXCR5, BMP2, BRCA1, BTLA, C3, CASP3, CASP9, CCL1, CCL11, CCL13, CCL16, CCL17, CCL18, CCL19, CCL2, CCL20, CCL21, CCL22, CCL23, CCL24, CCL25, CCL26, CCL27, CCL28, CCL3, CCL5, CCL7, CCL8, CCNB1, CCND1, CCNE1, CCR1, CCR10, CCR2, CCR3, CCR4, CCR5, CCR6, CCR7, CCR8, CCR9, CCRL2, CD154, CD19, CDla, CD2, CD226, CD244, PDCD1LG1, CD28, CD34, CD36, CD38, CD3E, CD3G, CD3Z, CD4, CD40LG, CD5, CD54, CD6, CD68, CD69, CLIP, CD80, CD83, SLAMF5, CD86, CD8A, CDH1, CDH7, CDK2, CDK4, CDKN1A, CDKN1B, CDKN2A, CDKN2B, CEACAM1, COL4A5, CREBBP, CRLF2, CSF1, CSF2, CSF3, CTLA4, CTNNB1, CTSC, CX3CL1, CX3CR1, CXCL1, CXCL10, CXCL11, CXCL12, CXCL13, CXCL14, CXCL16, CXCL2, CXCL3, CXCL5, CXCL6, CXCL9, CXCR3, CXCR4, CXCR6, CYP1A2, CYP7A1, DCC, DCN, DEFA6, DICERI, DKK1, Dok-1, Dok-2, DOK6, DVL1, E2F4, EBI3, ECE1, ECGF1, EDN1, EGF, EGFR, EIF4E, CD105, ENPEP, ERBB2, EREG, FCGR3A, CGR3B, FN1, FOXP3, FYN, FZD1, GAPD, GLI2, GNLY, GOLPH4, GRB2, GSK3B, GSTP1, GUSB, GZMA, GZMB, GZMH, GZMK, HLA-B, HLA-C, HLA-, MA, HLA-DMB, HLA-DOA, HLA-DOB, HLA-DPA1, HLA-DQA2, HLA-DRA, HLX1, HMOX1, HRAS, HSPB3, HUWE1, ICAM1, ICAM-2, ICOS, ID1, ifna1, ifna17, ifna2, ifna5, ifna6, ifna8, IFNAR1, IFNAR2, IFNG, IFNGR1, IFNGR2, IGF1, IHH, IKBKB, IL 10, IL12A, IL12B, IL 12RB1, IL 12RB2, IL 13, IL13RA2, IL 15, IL15RA, IL 17, IL17R, IL17RB, IL18, IL1A, IL1B, IL1R1, IL2, IL21, IL21R, IL23A, IL23R, IL24, IL27, IL2RA, IL2RB, IL2RG, IL3, IL31RA, IL4, IL4RA, IL5, IL6, IL7, IL7RA, IL8, CXCR1, CXCR2, IL9, IL9R, IRF1, ISGF3G, ITGA4, ITGA7, integrin alpha E (antigen CD103, human mucosal lymphocyte, antigen 1; alpha polypeptide), Gene hCG33203, ITGB3, JAK2, JAK3, KLRB1, KLRC4, KLRF1, KLRG1, KRAS, LAG3, LAIR2, LEF1, LGALS9, LILRB3, LRP2, LTA, SLAMF3, MADCAM1, MADH3, MADH7,MAF, MAP2K1, MDM2, MICA, MICB, MKI67, MMP12, MMP9, MTA1, MTSS1, MYC, MYD88, MYH6, NCAM1, NFATC1, NKG7, NLK, NOS2A, P2X7, PDCD1, PECAM-, CXCL4, PGK1, PIAS1, PIAS2, PIAS3, PIAS4, PLAT, PML, PP1A, CXCL7, PPP2CA, PRF1, PROM1, PSMB5, PTCH, PTGS2, PTP4A3, PTPN6, PTPRC, RAB23, RAC/RHO, RAC2, RAF, RB1, RBL1, REN, Drosha, SELE, SELL, SELP, SERPINE1, SFRP1, SIRP beta 1, SKI, SLAMF1, SLAMF6, SLAMF7, SLAMF8, SMAD2, SMAD4, SMO, SMOH, SMURF1, SOCS1, SOCS2, SOCS3, SOCS4, SOCS5, SOCS6, SOCS7, SOD1, SOD2, SOD3, SOS1, SOX17, CD43, ST14, STAM, STAT1, STAT2, STAT3, STAT4, STAT5A, STAT5B, STAT6, STK36, TAP1, TAP2, TBX21, TCF7, TERT, TFRC, TGFA, TGFB1, TGFBR1, TGFBR2, TIM-3, TLR1, TLR10, TLR2, TLR3, TLR4, TLR5, TLR6, TLR7, TLR8, TLR9, TNF, TNFRSF10A, TNFRSF11A, TNFRSF18, TNFRSF1A, TNFRSF1B, OX-40, TNFRSF5, TNFRSF6, TNFRSF7, TNFRSF8, TNFRSF9, TNFSF10, TNFSF6, TOB1, TP53, TSLP, VCAM1, VEGF, WIF1, WNT1, WNT4, XCL1, XCR1, ZAP70 and ZIC2.

In some embodiments, the immune markers are those described in WO2014023706 (incorporated by reference). Under this embodiment, an expression level EL₁ of a single gene representative of human adaptive immune response and of a single gene representative of human immunosuppressive response (a pair of genes) is assessed in the method of the invention.

In some embodiments, the gene representative of the adaptive immune response is selected from the cluster of the co-modulated genes for the Th1 adaptive immunity, for the cytotoxic response, or for the memory response, and may encode for a Th1 cell surface marker, an interleukin (or an interleukin receptor), or a chemokine or (a chemokine receptor). In some embodiments, the gene representative of the adaptive immune response is selected from the group consisting of:
the family of chemokines and chemokine receptors consisting of: CXCL13, CXCL9, CCL5, CCR2, CXCL10, CXCL 11, CXCR3, CCL2 and CX3CL1,
the family of cytokines consisting of: IL15,
the TH1 family consisting of: IFNG, IRF1, STAT1, STAT4 and TBX21
the family of lymphocytes membrane receptors consisting of: ITGAE, CD3D, CD3E, CD3G, CD8A, CD247, CD69 and ICOS,
the family of cytotoxic molecules consisting of: GNLY, GZMH, GZMA, GZMB, GZMK, GZMM and PRF1,
   and the kinase LTK.

In some embodiments, the gene representative of the adaptive immune response is selected from the group consisting of CCL5, CCR2, CD247, CD3E, CD3G, CD8A, CX3CL1, CXCL11, GZMA, GZMB, GZMH, GZMK, IFNG, IL15, IRF1, ITGAE, PRF1, STAT1 and TBX21.

In some embodiments, the gene representative of the adaptive immune response may be typically selected from the group of the co-modulated adaptive immune genes, whereas the immunosuppressive genes, may be representative of the inactivation of immune cells (e.g. dendritic cells) and may contribute to induction of an immunosuppressive response.

In some embodiments, the gene or corresponding proteins representative of the immunosuppressive response is selected from the group consisting of CD274, CTLA4, IHH, IL17A, PDCD1, PF4, PROM1, REN, TIM-3, TSLP, and VEGFA.

Under preferred conditions for implementing the invention, the gene representative of the adaptive immune response is selected from the group consisting of GNLY, CXCL13, CX3CL1, CXCL9, ITGAE, CCL5, GZMH, IFNG, CCR2, CD3D, CD3E, CD3G, CD8A, CXCL10, CXCL11, GZMA, GZMB, GZMK, GZMM, IL15, IRF1, LTK, PRF1, STAT1, CD69, CD247, ICOS, CXCR3, STAT4, CCL2 and TBX21 and the gene representative of the immunosuppressive response is selected from the group consisting of PF4, REN, VEGFA, TSLP, IL17A, PROM1, IIH, CD1A, CTLA4, PDCD1, CD276, CD274, TIM-3 and VTCN1 (B7H4).

Because some genes are more frequently found significant when combining one adaptive gene and one immunosuppressive gene, the most preferred genes are:
genes representative of the adaptive immune response: CD3G, CD8A, CCR2 and GZMA
genes representative of the immunosuppressive response: REN, IL17A, CTLA4 and PDCD1.

Under further preferred conditions for implementing the invention, a gene representative of the adaptive immune response and a gene representative of the immunosuppressive response are selected respectively from the groups consisting of the genes of Tables 1 and 2 above.

Preferred combinations of two pairs of genes (total of 4 genes) are
CCR2, CD3G, IL17A and REN and
CD8A, CCR2, REN and PDCD1.

In some embodiments, the immune markers indicative of the status of the immune response are those described in WO2014009535 (incorporated by reference). The immune markers indicative of the status of the immune response may comprise the expression level of one or more genes from the group consisting of CCR2, CD3D, CD3E, CD3G, CD8A, CXCL10, CXCL 11, GZMA, GZMB, GZMK, GZMM, IL15, IRF1, PRF1, STAT1, CD69, ICOS, CXCR3, STAT4, CCL2, and TBX21.

In some embodiments, the immune markers indicative of the status of the immune response are those described in WO2012095448 (incorporated by reference). The immune markers indicative of the status of the immune response may comprise the expression level of one or more genes from the group consisting of GZMH, IFNG, CXCL13, GNLY, LAG3, ITGAE, CCL5, CXCL9, PF4, IL17A, TSLP, REN, IHH, PROM1 and VEGFA.

In some embodiments, the immune markers indicative of the status of the immune response are those described in WO2012072750 (incorporated by reference). The immune markers indicative of the status of the immune response may comprise the expression level of a miRNA cluster comprising: miR.609, miR.518c, miR.520f, miR.220a, miR.362, miR.29a, miR.660, miR.603, miR.558, miR519b, miR.494, miR.130a, or miR.639.

In some embodiments, the immune response is assessed by a scoring system that inputs the quantification values of one or more immune markers as described above.

In some embodiments, the scoring system is a continuous scoring system. In some embodiments, the continuous scoring system inputs the absolute quantification values of one or more immune markers. In some embodiments, the continuous scoring system inputs the absolute quantification values of cell densities determined in the tumor biopsy sample obtained from the patient. In some embodiments, the continuous scoring system inputs the absolute quantification value of the CD3+ cell densities and the absolute quantification value of CD8+ cell densities. According to these embodiments, the scoring system outputs a continuous variable (i.e. score).

In some embodiments, the immune response is assessed by a continuous scoring system that involves the steps of:

a) quantifying one or more immune markers in a tumor biopsy sample obtained from said patient;

b) comparing each values obtained at step a) for said one or more immune markers with a distribution of values obtained for each of said one or more immune markers from a reference group of patients suffering from said cancer;

c) determining for each values obtained at step a) for said one or more immune markers the percentile of the distribution to which the values obtained at step a) correspond;

d) calculating the arithmetic mean value or the median value of percentile.

In some embodiments, the immune response is assessed by a continuous-scoring system that involves the steps of:

a) quantifying the density of CD3+ cells and the density of CD8+ cells in a tumor biopsy sample obtained from said patient;

b) comparing each density values obtained at step a) with a distribution of values obtained from a reference group of patients suffering from said cancer;

c) determining for each density values obtained at step a) the percentile of the distribution to which the values obtained at step a) correspond;

d) calculating the arithmetic mean value of percentile.

In some embodiments, the scoring system is a non-continuous system. In some embodiments, the scoring system is a non-continuous system wherein the absolute quantification values of one or more immune markers is assigned to a predetermined bin. In some embodiments, the scoring system is a non-continuous system wherein the absolute quantification values of cell densities determined in the tumor biopsy sample obtained from the patient are assigned to a “high” or “low” bin. In some embodiments, the scoring system is a non-continuous system wherein the absolute quantification values of CD3+ and CD8+ cell densities determined in the tumor biopsy sample obtained from the patient are assigned to a “high” or “low” bin. According to these particular embodiments, the cell density value is thus compared to a predetermined reference value and thus is assigned to a “low” or “high” bin depending on whether the cell density is lower or higher than the predetermined reference value. According to these embodiments, the scoring system outputs a non-continuous variable such as “low”, “medium” and “high”.

In some embodiments, the immune response is assessed by a non-continuous scoring system that involves the steps of.

a) quantifying one or more immune markers in a tumor biopsy sample obtained from said patient;

b) comparing each values obtained at step a) for said one or more immune markers with a distribution of values obtained for each of said one or more immune markers from a reference group of patients suffering from said cancer;

c) determining for each values obtained at step a) for said one or more immune markers the percentile of the distribution to which the values obtained at step a) correspond;

d) calculating the arithmetic mean value or the median value of percentile; and

e) comparing the arithmetic mean value or the median value of percentile obtained at step d) with a predetermined reference arithmetic mean value or a predetermined median value of percentile, and

f) assigning a “low” or “high” score depending on whether the arithmetic mean value or the median value of percentile is respectively lower or higher than the predetermined reference arithmetic mean value or a predetermined median value of percentile.

In some embodiments, the immune response is assessed by a scoring system that involves the steps of:

a) quantifying the density of CD3+ cells and the density of CD8+ cells in a tumor biopsy sample obtained from said patient;

b) comparing each density values obtained at step a) with a distribution of values obtained from a reference group of patients suffering from said cancer;

c) determining for each density values obtained at step a) the percentile of the distribution to which the values obtained at step a) correspond;

d) calculating the arithmetic mean value of percentile; and

e) comparing the arithmetic mean value obtained at step d) with a predetermined reference arithmetic mean value of percentile, and

f) assigning a “low” or “high” score depending on whether the arithmetic mean value of percentile is respectively lower or higher than the predetermined reference arithmetic mean value of percentile.

In some embodiments, the immune response is assessed by a scoring system that involves the steps of:

a) quantifying the density of CD3+ cells and the density of CD8+ cells in a tumor biopsy sample obtained from said patient;

b) comparing each density values obtained at step a) with a distribution of values obtained from a reference group of patients suffering from said cancer;

c) determining for each density values obtained at step a) the percentile of the distribution to which the values obtained at step a) correspond;

d) calculating the arithmetic mean value of percentile; and

e) comparing the arithmetic mean value of percentile obtained at step d) with 2 predetermined reference arithmetic mean values percentile, and

f) assigning a “low” “intermediate” or “high” score depending on whether the arithmetic mean value:
is lower than the lowest predetermined reference arithmetic mean value of percentile (“low”)
is comprised between the 2 predetermined reference arithmetic mean values of percentile (“intermediate”)
is higher than the highest predetermined reference arithmetic mean value of percentile (“high”).

In some embodiments, the non-continuous scoring system is Immunoscore as described in the EXAMPLE.

In some embodiments, the scoring system for assessing the immune response involves digital pathology as described herein after and in the EXAMPLE.

In some embodiments, the scoring system is an automated scoring system.

Methods for Quantifying the Immune Marker:

Any one of the methods known by the one skilled in the art for quantifying cellular types, a protein-type or a nucleic acid-type immune marker encompassed herein may be used for performing the cancer prognosis method of the invention. Thus any one of the standard and non-standard (emerging) techniques well known in the art for detecting and quantifying a protein or a nucleic acid in a sample can readily be applied.

Expression of an immune marker of the invention may be assessed by any of a wide variety of well-known methods for detecting expression of a transcribed nucleic acid or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In some embodiments, expression of a marker is assessed using an antibody (e.g. a radio-labeled, chromophore-labeled, fluorophore-labeled, polymer-backbone-antibody, or enzyme-labeled antibody), an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with a marker protein or fragment thereof, including a marker protein which has undergone all or a portion of its normal post-translational modification.

In some embodiments, an immune marker, or a set of immune markers, may be quantified with any one of the immunohistochemistry methods known in the art.

Typically, for further analysis, one thin section of the tumor, is firstly incubated with labeled antibodies directed against one immune marker of interest. After washing, the labeled antibodies that are bound to said immune marker of interest are revealed by the appropriate technique, depending of the kind of label is borne by the labeled antibody, e.g. radioactive, fluorescent or enzyme label. Multiple labelling can be performed simultaneously.

Immunohistochemistry typically includes the following steps i) fixing the tumor biopsy sample with formalin, ii) embedding said tumor biopsy sample in paraffin, iii) cutting said tumor biopsy sample into sections for staining, iv) incubating said sections with the binding partner specific for the immune marker, v) rinsing said sections, vi) incubating said section with a secondary antibody typically biotinylated and vii) revealing the antigen-antibody complex typically with avidin-biotin-peroxidase complex. Accordingly, the tumor biopsy sample is firstly incubated with the binding partners having for the immune marker. After washing, the labeled antibodies that are bound to the immune marker are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. Hematoxylin & Eosin, DAPI, Hoechst. Other staining methods may be accomplished using any suitable method or system as would be apparent to one of skill in the art, including automated, semi-automated or manual systems.

For example, one or more labels can be attached to the antibody, thereby permitting detection of the target protein (i.e. the immune markers). Exemplary labels include radioactive isotopes, fluorophores, ligands, chemiluminescent agents, enzymes, and combinations thereof. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. ³H, ¹⁴C, ³²P, ³⁵S or ¹²⁵I) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In some embodiments, the label is a quantum dot. For example, Quantum dots (Qdots) are becoming increasingly useful in a growing list of applications including immunohistochemistry, flow cytometry, and plate-based assays, and may therefore be used in conjunction with this invention. Qdot nanocrystals have unique optical properties including an extremely bright signal for sensitivity and quantitation; high photostability for imaging and analysis. A single excitation source is needed, and a growing range of conjugates makes them useful in a wide range of cell-based applications. Qdot Bioconjugates are characterized by quantum yields comparable to the brightest traditional dyes available. Additionally, these quantum dot-based fluorophores absorb 10-1000 times more light than traditional dyes. The emission from the underlying Qdot quantum dots is narrow and symmetric which means overlap with other colors is minimized, resulting in minimal bleed through into adjacent detection channels and attenuated crosstalk, in spite of the fact that many more colors can be used simultaneously. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled.

In some embodiments, the resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the immune checkpoint protein in the sample, or the absolute number of cells positive for the maker of interest, or the surface of cells positive for the maker of interest. Various automated sample processing, scanning and analysis systems suitable for use with IHC are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantify the presence of the specified biomarker (i.e. immune checkpoint protein). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms and tissue recognition pattern (e.g. Aperio Spectrum Software, Automated QUantitatative Analysis platform (AQUA® platform), or Tribvn with Ilastic and Calopix software), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the immune checkpoint protein) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. For example, the amount can be quantified as an absolute number of cells positive for the maker of interest. In some embodiments, a score is given to the sample that is a numerical representation of the intensity or amount of the histochemical staining of the sample, and represents the amount of target biomarker (e.g., the immune checkpoint protein) present in the sample. Optical density or percentage area values can be given a scaled score, for example on an integer scale.

Thus, in some embodiments, the method of the present invention comprises the steps consisting in i) providing one or more immunostained slices of tissue section obtained by an automated slide-staining system by using a binding partner capable of selectively interacting with the immune marker, ii) proceeding to digitalisation of the slides of step i).by high resolution scan capture, iii) detecting the slice of tissue section on the digital picture iv) providing a size reference grid with uniformly distributed units having a same surface, said grid being adapted to the size of the tissue section to be analysed, and v) detecting, quantifying and measuring intensity or the absolute number of stained cells in each unit.

Multiplex tissue analysis techniques are particularly useful for quantifying several immune checkpoint proteins in the tumor biopsy sample. Such techniques should permit at least five, or at least ten or more biomarkers to be measured from a single tumor biopsy sample. Furthermore, it is advantageous for the technique to preserve the localization of the biomarker and be capable of distinguishing the presence of biomarkers in cancerous and non-cancerous cells. Such methods include layered immunohistochemistry (L-IHC), layered expression scanning (LES) or multiplex tissue immunoblotting (MTI) taught, for example, in U.S. Pat. Nos. 6,602,661, 6,969,615, 7,214,477 and 7,838,222; U.S. Publ. No. 2011/0306514 (incorporated herein by reference); and in Chung & Hewitt, Meth Mol Biol, Prot Blotting Detect, Kurlen & Scofield, eds. 536: 139-148, 2009, each reference teaches making up to 8, up to 9, up to 10, up to 11 or more images of a tissue section on layered and blotted membranes, papers, filters and the like, can be used. Coated membranes useful for conducting the L-IHC/MTI process are available from 20/20 GeneSystems, Inc. (Rockville, Md.).

In some embodiments, the L-IHC method can be performed on any of a variety of tissue samples, whether fresh or preserved. The samples included core needle biopsies that were routinely fixed in 10% normal buffered formalin and processed in the pathology department. Standard five m thick tissue sections were cut from the tissue blocks onto charged slides that were used for L-IHC. Thus, L-IHC enables testing of multiple markers in a tissue section by obtaining copies of molecules transferred from the tissue section to plural bioaffinity-coated membranes to essentially produce copies of tissue “images.” In the case of a paraffin section, the tissue section is deparaffinized as known in the art, for example, exposing the section to xylene or a xylene substitute such as NEO-CLEAR®, and graded ethanol solutions. The section can be treated with a proteinase, such as, papain, trypsin, proteinase K and the like. Then, a stack of a membrane substrate comprising, for example, plural sheets of a 10 m thick coated polymer backbone with 0.4 m diameter pores to channel tissue molecules, such as, proteins, through the stack, then is placed on the tissue section. The movement of fluid and tissue molecules is configured to be essentially perpendicular to the membrane surface. The sandwich of the section, membranes, spacer papers, absorbent papers, weight and so on can be exposed to heat to facilitate movement of molecules from the tissue into the membrane stack. A portion of the proteins of the tissue are captured on each of the bioaffinity-coated membranes of the stack (available from 20/20 GeneSystems, Inc., Rockville, Md.). Thus, each membrane comprises a copy of the tissue and can be probed for a different biomarker using standard immunoblotting techniques, which enables open-ended expansion of a marker profile as performed on a single tissue section. As the amount of protein can be lower on membranes more distal in the stack from the tissue, which can arise, for example, on different amounts of molecules in the tissue sample, different mobility of molecules released from the tissue sample, different binding affinity of the molecules to the membranes, length of transfer and so on, normalization of values, running controls, assessing transferred levels of tissue molecules and the like can be included in the procedure to correct for changes that occur within, between and among membranes and to enable a direct comparison of information within, between and among membranes. Hence, total protein can be determined per membrane using, for example, any means for quantifying protein, such as, biotinylating available molecules, such as, proteins, using a standard reagent and method, and then revealing the bound biotin by exposing the membrane to a labeled avidin or streptavidin; a protein stain, such as, Blot fastStain, Ponceau Red, brilliant blue stains and so on, as known in the art.

In some embodiments, the present methods utilize Multiplex Tissue Imprinting (MTI) technology for measuring biomarkers, wherein the method conserves precious biopsy tissue by allowing multiple biomarkers, in some cases at least six biomarkers.

In some embodiments, alternative multiplex tissue analysis systems exist that may also be employed as part of the present invention. One such technique is the mass spectrometry-based Selected Reaction Monitoring (SRM) assay system (“Liquid Tissue” available from OncoPlexDx (Rockville, Md.)). That technique is described in U.S. Pat. No. 7,473,532.

In some embodiments, the method of the present invention utilized the multiplex IHC technique developed by GE Global Research (Niskayuna, N.Y.). That technique is described in U.S. Pub. Nos. 2008/0118916 and 2008/0118934. There, sequential analysis is performed on biological samples containing multiple targets including the steps of binding a fluorescent probe to the sample followed by signal detection, then inactivation of the probe followed by binding probe to another target, detection and inactivation, and continuing this process until all targets have been detected.

In some embodiments, multiplex tissue imaging can be performed when using fluorescence (e.g. fluorophore or Quantum dots) where the signal can be measured with a multispectral imagine system. Multispectral imaging is a technique in which spectroscopic information at each pixel of an image is gathered and the resulting data analysed with spectral image-processing software. For example, the system can take a series of images at different wavelengths that are electronically and continuously selectable and then utilized with an analysis program designed for handling such data. The system can thus be able to obtain quantitative information from multiple dyes simultaneously, even when the spectra of the dyes are highly overlapping or when they are co-localized, or occurring at the same point in the sample, provided that the spectral curves are different. Many biological materials auto fluoresce, or emit lower-energy light when excited by higher-energy light. This signal can result in lower contrast images and data. High-sensitivity cameras without multispectral imaging capability only increase the autofluorescence signal along with the fluorescence signal. Multispectral imaging can unmix, or separate out, autofluorescence from tissue and, thereby, increase the achievable signal-to-noise ratio. Briefly the quantification can be performed by following steps: i) providing a tumor tissue microarray (TMA) obtained from the patient, ii) TMA samples are then stained with anti-antibodies having specificity of the immune checkpoint protein(s) of interest, iii) the TMA slide is further stained with an epithelial cell marker to assist in automated segmentation of tumour and stroma, iv) the TMA slide is then scanned using a multispectral imaging system, v) the scanned images are processed using an automated image analysis software (e.g.Perkin Elmer Technology) which allows the detection, quantification and segmentation of specific tissues through powerful pattern recognition algorithms. The machine-learning algorithm was typically previously trained to segment tumor from stroma and identify cells labelled.

Determining an expression level of a gene in a tumor sample obtained from a patient can be implemented by a panel of techniques well known in the art.

In some embodiments, an expression level of a gene is assessed by determining the quantity of mRNA produced by this gene.

Methods for determining a quantity of mRNA are well known in the art. For example nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The thus extracted mRNA is then detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of Amplification include ligase chain reaction (LCR), transcription-mediated amplification (TMA), strand displacement amplification (SDA) and nucleic acid sequence based amplification (NASBA), quantitative new generation sequencing of RNA (NGS).

Nucleic acids (s) comprising at least 10 nucleotides and exhibiting sequence complementarity or homology to the mRNA of interest herein find utility as hybridization probes or amplification primers. It is understood that such nucleic acids need not be completely identical, but are typically at least about 80% identical to the homologous region of comparable size, more preferably 85% identical and even more preferably 90-95% identical. In some embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin). Probes typically comprise single-stranded nucleic acids of between 10 to 1000 nucleotides in length, for instance of between 10 and 800, more preferably of between 15 and 700, typically of between 20 and 500 nucleotides. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

Nucleic acids which may be used as primers or probes in the above amplification and detection method may be assembled as a kit. Such a kit includes consensus primers and molecular probes. A preferred kit also includes the components necessary to determine if amplification has occurred. A kit may also include, for example, PCR buffers and enzymes; positive control sequences, reaction control primers; and instructions for amplifying and detecting the specific sequences.

In some embodiments, the expression of an immune marker of the invention may be assessed by tagging the biomarker (in its DNA, RNA or protein for) with a digital oligonucleotide barcode, and to measure or count the number of barcodes.

In some embodiments, the methods of the invention comprise the steps of providing total RNAs extracted from cumulus cells and subjecting the RNAs to amplification and hybridization to specific probes, more particularly by means of a quantitative or semi-quantitative RT-PCR. Probes made using the disclosed methods can be used for nucleic acid detection, such as in situ hybridization (ISH) procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pinkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. J. Pathol. 157:1467-1472, 2000 and U.S. Pat. No. 6,942,970. Additional detection methods are provided in U.S. Pat. No. 6,280,929. Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publications Nos. 2006/0246524; 2006/0246523, and 2007/0117153.

It will be appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In some embodiments, the methods of the invention comprise the steps of providing total RNAs extracted from cumulus cells and subjecting the RNAs to amplification and hybridization to specific probes, more particularly by means of a quantitative or semi-quantitative RT-PCR. In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

The expression level of a gene may be expressed as absolute expression level or normalized expression level. Both types of values may be used in the present method. The expression level of a gene is preferably expressed as normalized expression level when quantitative PCR is used as method of assessment of the expression level because small differences at the beginning of an experiment could provide huge differences after a number of cycles.

In some embodiments, the nCounter® Analysis system is used to detect intrinsic gene expression. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-over-lapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the tumor tissue sample with a probe library, such that the presence of the target in the sample creates a probe pair—target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe,electrophoresed to elongate the reporter probes, and immobilized. After processing, the sample cartridge is transferred to a fully automated imaging and data collection device (Digital Analyzer, NanoString Technologies). The level of a target is measured by imaging each sample and counting the number of times the code for that target is detected. For each sample, typically 600 fields-of-view (FOV) are imaged (1376×1024 pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

Typically, expression levels are normalized by correcting the absolute expression level of a gene by comparing its expression to the expression of a gene that is not relevant for determining the cancer stage of the patient, e.g., a housekeeping gene that is constitutively expressed. Suitable genes for normalization include housekeeping genes such as the actin gene ACTB, ribosomal 18S gene, GUSB, PGK1 and TFRC. This normalization allows comparing the expression level of one sample, e.g., a patient sample, with the expression level of another sample, or comparing samples from different sources.

Assessment of the Clinical Response after the Preoperative Adjuvant Therapy:

In some embodiments, the clinical response after the preoperative adjuvant therapy is assessed by any well-known method in the art. Typically, the clinical response is assessed by imaging (such as CT scan, MRI, IRM echo scan), biopsy, liquid biopsy, cytology, level of tumoral biomarker marker measured in circulating blood, level of ctDNA, levels of circulating tumour cells, and/or pathological analysis of resected tumor.

In some embodiments, the clinical response is determined by the assessment of the level of ctDNA. Methods for determining the level of ctDNA are well known in the art. For example, the method is described in WO2012/028746. Q-PCR is thus the preferred method for determining said level. In some embodiment, the method comprises the step of amplifying and quantifying a nuclear target nucleic acid sequence According to the invention, the nuclear target nucleic acid sequence is a sequence which is located in the nucleus human genome. The skilled person can thus easily select the appropriate nuclear target nucleic acid sequences. Cell free nucleic acid in a patient suffering from a cancer is constituted of nucleic acids of tumor and non-tumor origin. Thus in some embodiments, the nuclear target nucleic sequence is a mutant target nucleic acid sequence, i.e. a nucleic acid bearing a tumoral mutation of interest. It is thus important to select a mutation which has a tumor origin to quantify only the nucleic acids which derives from cancer cells. In some embodiments, the mutation is located in a the KRAS gene or TP53 gene. For example, the mutation is located in a gene selected from the group consisting of TP53 (394, 395, 451, 453, 455, 469, 517, 524, 527, 530, 586, 590, 637, 641, 724, 733, 734, 743, 744, 817, 818, 819, 820, 839, 844, 916) or PIK3CA (1530, 1624, 1633, 1634, 1636, 1656, 3140, 3140, 3140). KRAS mutation may include any mutation as described above. Typically, the target nucleic acid sequences have a length between 160 and 210 base pairs. In some embodiments, the target nucleic acid sequence has a length of 160; 165; 170; 175; 180; 185; 190; 195; 200; 205; or 210 base pairs.

In some embodiments, the clinical response is determined by the assessment of reduction in tumor volume that has been used as the standard criterion for response evaluation among solid tumors. In some embodiments, the reduction in tumor volume is assessed by imaging.

In some embodiments, the clinical response is assessed by radiography. For instance, the reduction in tumor volume is assessed by mammography for a patient suffering from breast cancer.

In some embodiments, the clinical response is assessed by ultrasound imaging. In some embodiments, the ultrasound imaging is a three-dimensional ultrasound imaging. In some embodiments, ultrasound imaging involves the use of primary color Doppler. Doppler ultrasound vascularization includes evaluation of parameters such as: the number of signs of flow, a peak flow velocity, the resistivity index (RI) and pulsatility index (PI). It also allows the noninvasive evaluation of abnormal vessel architecture in tumors, referred to as neoangiogenesis. These changes in vascularization of the tumor correlate to the histopathological response, and therefore, the study of vascularization can be used as a complementary tool to assess the response to preoperative adjuvant therapy, primarily in locally advanced breast cancer.

In some embodiments, the clinical response is assessed by magnetic resonance. Magnetic resonance imaging increases the accuracy of radiography evaluated in monitoring response to preoperative adjuvant therapy, and therefore it is relevant in the evaluation of a possible conservative surgery.

In some embodiments, the clinical response is assessed by scintigraphy. In some embodiments, the chelated metal is Tc, In, Ga. Y, Lu, Re, Cu, Ac, Bi, Pb, Sm, Sc, Co, Ho, Gd, Eu, Tb, or Dy. In some embodiments the metal is an isotope, for example a radioactive isotope In some embodiments, the isotope is Tc-94m, Tc-99m, In-1 11, G-67, Ga-68, Y-86, Y-90, Lu-177, Re-186, Re-188, Cu-64, Cu-67, Co-55, Co-57, Sc-47, Ac-225, Bi-213, Bi-212, Pb-212, Sm-153, Ho-166, or Dy-166. In some embodiments, scintigraphy involves use of technetium (99mTc) sestamibi that is a pharmaceutical agent used in nuclear medicine imaging. The drug is a coordination complex consisting of the radioisotope technetium-99m bound to six (sesta=6) methoxyisobutylisonitrile (MIBI) ligands. Mechanisms of accumulation and efflux of 99mTc-sestamibi in carcinomas involve cellular processes that are important in tumor response to treatment.

In some embodiments, the clinical response is assessed by Tomography Emission Positron/Computed Tomography (PET/CT). Positron Emission Tomography (PET) is a powerful technique to image biochemical or physiological processes within the body. The metabolic and biological activity of disease always precedes any anatomic evidence of the illness. PET is a biological imaging technique does not replace anatomical imaging as X Ray, computed tomography (CT) or magnetic resonance imaging (MRI), but adds the characterization of simple molecular process that are taking place in normal or diseased tissues within the body. Positron is an antiparticle of the electron. When it is spelled from the nucleous of an atom, it travel only a short distance. During this travel across several millimeters, adjacent atoms are ionized and the positron loses energy and slow down. Positron then pairs up with an electron and undergoing an annihilation interaction, which produces a pair of 511 KeV annihilation photons that travel in opposite directions reaching PET radiation detectors for imaging. The fusion of PET and CT images is very useful in the correlation of the exact site of anatomical and physiological information. In PET oncologic imaging, the most widely radiopharmaceutical is 2-[18F]-fluoro-2-deoxy-D-glucose (18F-FDG). Biochemically 18F-FDG is a nonphysiological compound with a chemical structure very similar to that of naturally occurring glucose; it serves as an external marker of cellular glucose metabolism. The ability no noninvasively image cellular glucose metabolism is important in oncological applications because many cancer cells use glucose at higher rates. The absolute quantitative radiotracer uptake in tumor can be measured in an effort to differentiate between malignant and benign tissue. Named Standard Uptake Value (SUV) it can be useful in measuring tumor metabolic function.

In some embodiments, the reduction in tumor volume is appreciated by aligning the images of the tumor before and after the preoperative adjuvant therapy. In some embodiments, aligning the images allows for quantification of change on a pixel-by-pixel or voxel-by-voxel basis which can be used to provide a very sensitive approach to detect, quantify and spatially display changes in image contrasts to provide for detailed insight into the status, extent, progression and therefore clinical response using images obtained from a wide variety of imaging modalities, for example, but not limited to magnetic resonance imaging (MRI), computed tomography (CT), two-dimensional planar X-Ray, positron emission tomography (PET), ultrasound (US), optical imaging (i.e. fluorescence, near-infrared (NIR) & bioluminescence), and single-photon emission computed tomography (SPECT). Within a given instrumentation source (e.g. MRI, CT, X-Ray, PET and SPECT) a variety of data can be generated. For example, MRI devices can generate diffusion, perfusion, permeability, normalized and spectroscopic images, which include molecules containing, for example, but not limited to, ¹H, ¹³C, ²³-Na, ³¹P, and ¹⁹F, hyperpolarized Helium, Xenon and/or ¹³C MRI, which can also be used to generate kinetic parameter maps. PET, SPECT and CT devices are also capable of generating static images as well as kinetic parameters by fitting temporally resolved imaging data to a pharmacokinetic model. Imaging data, irrespective of source and modality, can be presented as quantified (i.e., has physical units) or normalized (i.e., images are normalized to an external phantom or something of known and constant property or a defined signal within the image volume) maps so that images can be compared between patients as well as data acquired during different scanning sessions.

In some embodiments, the clinical response is assessed by a scoring system. In some embodiments, the clinical response is assessed by a non-continuous scoring system. In some embodiments, the clinical response is assessed by the ycTNM scoring system. In some embodiments, a complete clinical response (cCR) is considered as achieved when the original mass became undetectable. In some embodiments, a partial response (cPR) represents a 50% or greater reduction in bi-dimensional tumor measurements. In some embodiments, a progressive disease (cPD) is detected if bi-dimensional measurements increased by 20% or more. In some embodiments, all others clinical response are classified as stable disease (cSD).

Uses of Algorithms:

In some embodiments, the method of the present invention involves use of an algorithm.

In some embodiments, the method of the present invention comprises the steps of:

a) assessing at least two parameters, wherein the first parameter is the immune response determined before the preoperative adjuvant therapy and the second parameter is the clinical response determined after the preoperative adjuvant therapy

b) implementing an algorithm on data comprising or consisting of the parameters assessed at step a) as to obtain an algorithm output, the implementing step being computer-implemented; and

c) determining the risk of recurrence and/or death from the algorithm output obtained at step b).

Non-limiting examples of algorithms include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations. Non-limiting examples of algorithms thus include logistic regression, linear regression, random forests, classification and regression trees (C&RT), boosted trees, neural networks (NN), artificial neural networks (ANN), neuro fuzzy networks (NFN), network structures, perceptrons such as multi-layer perceptrons, multi-layer feed-forward networks, support vector machines (e.g., Kernel methods), multivariate adaptive regression splines (MARS), Levenberg-Marquardt algorithms, Gauss-Newton algorithms, mixtures of Gaussians, gradient descent algorithms, learning vector quantization (LVQ), and combinations thereof. Of particular use in combining parameters are linear and non-linear equations and statistical classification analyses to determine the relationship between levels of said parameters and the objective response to the preoperative adjuvant therapy. Of particular interest are structural and syntactic statistical classification algorithms, and methods of risk index construction, utilizing pattern recognition features, including established techniques such as cross-correlation, Principal Components Analysis (PCA), factor rotation, Logistic Regression (Log Reg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, Support Vector Machines, and Hidden Markov Models, among others. Other techniques may be used in survival and time to event hazard analysis, including Cox, Weibull, Kaplan-Meier and Greenwood models well known to those of skill in the art.

In some embodiments, the method of the present invention comprises the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models. The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering. In some embodiments, the machine learning algorithms comprise a reinforcement learning algorithm Examples of reinforcement learning algorithms include, but are not limited to, temporal difference learning, Q-learning and Learning Automata. Alternatively, the machine learning algorithm may comprise Data Pre-processing.

In some embodiments, the algorithm is implemented on a computer using well-known computer processors, memory units, storage devices, computer software, and other components. Typically, the computer contains a processor, which controls the overall operation of the computer by executing computer program instructions which define such operation. The computer program instructions may be stored in a storage device (e.g., magnetic disk) and loaded into memory when execution of the computer program instructions is desired. The computer also includes other input/output devices that enable user interaction with the computer (e.g., display, keyboard, mouse, speakers, buttons, etc.). One skilled in the art will recognize that an implementation of an actual computer could contain other components as well.

In some embodiments, the algorithm is implemented using computers operating in a client-server relationship. Typically, in such a system, the client computers are located remotely from the server computer and interact via a network. The client-server relationship may be defined and controlled by computer programs running on the respective client and server computers. In some embodiments, the results may be displayed on the system for display, such as with LEDs or an LCD. Accordingly, in some embodiments, the algorithm can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet. The computing system can include clients and servers. A client and server are generally remote from each other and typically interact through a communication network. The relationship of client and server arises by virtue of computer programs running on the respective computers and having a client-server relationship to each other.

In some embodiments, the algorithm is implemented within a network-based cloud computing system. In such a network-based cloud computing system, a server or another processor that is connected to a network communicates with one or more client computers via a network. A client computer (e.g. a mobile device, such as a phone, tablet, or laptop computer) may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For instance, the physician may register the parameters (i.e. input data) on, which then transmits the data over a long-range communications link, such as a wide area network (WAN) through the Internet to a server with a data analysis module that will implement the algorithm and finally return the output (e.g. score) to the mobile device.

In some embodiments, the output results can be incorporated in a Clinical Decision Support (CDS) system. These output results can be integrated into an Electronic Medical Record (EMR) system.

In other words, the interaction between a computer program product and the system enables to carry out the method of the invention. The method of the invention is thus a computer-implemented method. This means that the method is, at least partly computer-implemented. In particular, each step can be computer-implemented provided some steps are achieved by receiving data.

The system is a desktop computer. In variant, the system is a rack-mounted computer, a laptop computer, a tablet computer, a Personal Digital Assistant (PDA) or a smartphone.

In some embodiments, the computer is adapted to operate in real-time and/or is an embedded system, notably in a vehicle such as a plane. In the present case, the system comprises a calculator, a user interface and a communication device. The calculator is electronic circuitry adapted to manipulate and/or transform data represented by electronic or physical quantities in registers of the system X and/or memories in other similar data corresponding to physical data in the memories of the registers or other kinds of displaying devices, transmitting devices or memory devices. As specific examples, the calculator comprises a monocore or multicore processor (such as a central processing unit (CPU), a graphics processing unit (GPU), a microcontroller and a Digital Signal Processor (DSP)), a programmable logic circuitry (such as an Application Specific Integrated Circuit (ASIC), a Field Programmable Gate Array (FPGA), a Programmable Logic Device (PLD) and programmable logic arrays (PLA)), a state machine, gated logic and discrete hardware components. The calculator comprises a data-processing unit which is adapted to process data, notably by carrying out calculations, memories adapted to store data and a reader adapted to read a computer readable medium. The user interface comprises an input device and an output device. The input device is a device enabling the user of the system to input information or command to the system. In the present case, the input device is a keyboard. Alternatively, the input device is a pointing device (such as a mouse, a touch pad and a digitizing tablet), a voice-recognition device, an eye tracker or a haptic device (motion gestures analysis). The output device is a graphical user interface, that is a display unit adapted to provide information to the user of the system. In the present case, the output device is a display screen for visual presentation of output. In other embodiments, the output device is a printer, an augmented and/or virtual display unit, a speaker or another sound generating device for audible presentation of output, a unit producing vibrations and/or odors or a unit adapted to produce electrical signal.

In some embodiments, the input device and the output device are the same component forming man-machine interfaces, such as an interactive screen.

The communication device enables unidirectional or bidirectional communication between the components of the system. For instance, the communication device is a bus communication system or a input/output interfaces.

The presence of the communication device enables that, in some embodiments, the components of the calculator be remote one from another.

The computer program product comprises a computer readable medium. The computer readable medium is a tangible device that can be read by the reader of the calculator. Notably, the computer readable medium is not a transitory signal per se, such as radio waves or other freely propagating electromagnetic waves, such as light pulses or electronic signals. Such computer readable storage medium is, for instance, an electronic storage device, a magnetic storage device, an optical storage device, an electromagnetic storage device, a semiconductor storage device or any combination thereof. As a non-exhaustive list of more specific examples, the computer readable storage medium is a mechanically encoded device such a punchcards or raised structures in a groove, a diskette, a hard disk, a read-only memory (ROM), a random access memory (RAM), an erasable programmable read-only memory (EROM), electrically erasable and programmable read only memory (EEPROM), a magnetic-optical disk, a static random access memory (SRAM), a compact disc read-only memory (CD-ROM), a digital versatile disk (DVD), a memory stick, a floppy disk, a flash memory, a solid state drive disk (SSD) or a PC card such as a Personal Computer Memory Card International Association (PCMCIA).

In some embodiments, the computer program is stored in the computer readable storage medium. The computer program comprises one or more stored sequence of program instructions. Such program instructions when run by the data-processing unit, cause the execution of steps of the method of the invention. For instance, the form of the program instructions is a source code form, a computer executable form or any intermediate forms between a source code and a computer executable form, such as the form resulting from the conversion of the source code via an interpreter, an assembler, a compiler, a linker or a locator. In variant, program instructions are a microcode, firmware instructions, state-setting data, configuration data for integrated circuitry (for instance VHDL) or an object code. Typically, program instructions are written in any combination of one or more languages, such as an object oriented programming language (FORTRAN, C++, JAVA, HTML), procedural programming language (language C for instance).

In some embodiments, the program instructions are downloaded from an external source through a network as it is notably the case for applications. In such case, the computer program product comprises a computer-readable data carrier having stored thereon the program instructions or a data carrier signal having encoded thereon the program instructions. In each case, the computer program product comprises instructions which are loadable into the data-processing unit and adapted to cause execution of the method of the invention when run by the data-processing unit. According to the embodiments, the execution is entirely or partially achieved either on the system, that is a single computer, or in a distributed system among several computers (notably via cloud computing).

In some embodiments, the above-described method is implemented in many ways, notably using hardware, software or a combination thereof. In particular, each step is implemented by a module adapted to achieve the step or computer instructions adapted to cause the execution of the step by interaction with the system or a specific apparatus comprising the system. It should also be noted that two steps in succession may, in fact, be executed substantially concurrently or in a reverse order depending on the considered embodiments.

### Applications of the Method of the Present Invention

The method of the present invention is particularly suitable for orientating the clinical decisions after the preoperative adjuvant therapy.

In some embodiments, when it is concluded that the patient is will have a low risk of recurrence and/or death, an organ preservation strategy may be decided. Standard use of preoperative adjuvant therapy, tumor excision, and postoperative adjuvant therapy in locally advanced cancer has tremendously improved oncologic outcomes over the past several decades. However, these improvements come with costs of significant morbidity and poor quality of life. The method of the present invention provides the advantage of identifying a certain subgroup of patients that have exceptionally good clinical outcomes while preserving quality of life. Driven by patient demand and interest in preserving quality of life, the method of the present invention provides a powerful tool for implementing an organ preservation strategy as well as a watch and wait strategy so that the quality of life of the responder patient may be preserved. More particularly, the method of the present invention is particularly suitable for managing the risk of perioperative morbidity and mortality, particularly for the elderly. More particularly, the method of the present invention is particularly suitable for preventing ample loss of anorectal, sexual, and urinary function, which eventually leads to poor quality of life in patients suffering from colorectal cancer.

In particular, when the clinical response is ycTNM=0-I, the higher is the Immunoscore (e.g. the arithmetic mean or median value of percentile), the lower will be the risk of recurrence and/or death, and the longer will be the survival time (e.g. disease-free survival time) of the patient, and thus an organ preservation strategy may thus be decided.

In particular, when it is determined that the clinical response is ycTNM=0-I, and the Immunoscore was classified as “high” (e.g. the arithmetic mean or median value of percentile was classified as “high”) it is concluded that the patient will have a low risk of recurrence and/or death, and thus the survival time of the patient will be longer, and thus an organ preservation strategy may thus be decided.

In some embodiments, when it is concluded that the patient will have a high risk of recurrence and/or death and thus a short survival time, a radical surgery and adjuvant therapy is then decided.

The method of the present invention is thus particularly suitable for determining whether or not the patient is eligible to radical surgery after the preoperative adjuvant therapy.

The invention will be further illustrated by the following figures and examples. However, these examples and figures should not be interpreted in any way as limiting the scope of the present invention.

### FIGURES

FIG. 1. Disease-free survival according to IS_{B} Low, IS_{B} Intermediate (Int.), and IS_{B} High in patients with A) ycTNM=0 or I and B) ycTNM=II, III or IV, as determined by imaging post-neoajuvant therapy. P test for trend (P (tft)) is determined by log rank test for trend.

FIG. 2. Probability of the 2-year and 5-year Disease-free survival according to IS_{B} expressed as a continuous variable (IS_{B} mean score, in percentiles) in patients with A) ycTNM=0 or I and B) ycTNM=II, III or IV, as determined by imaging post-neoajuvant therapy, under the Cox proportional hazards regression model.

FIG. 3. Forest plots for Disease-free survival illustrating the hazard ratio according to IS_{B} Low vs Intermediate (Int.) and Low vs High, in patients with ycTNM=0 or I and ycTNM=II, III or IV, as determined by imaging post-neoajuvant therapy.

### EXAMPLE

Patients and Methods:

Patient Population

Two retrospective consecutive cohorts of LARC patients (n₁=131, n₂=118) with available biopsies, treated by nT and radical surgery by total mesorectal excision (TME) were analyzed. Cohort 1 was a monocentric cohort and cohort 2 was multicentric (Table 1). Inclusion period ranged from 1999 to 2016. Neoadjuvant treatment and surgery criteria were defined by each institution. Overall, 64.2% of patients were male and the median age at diagnosis was 65 (interquartile range [IQR]=53.3-74.1). Patients were treated by nT (short [3.7%] or long [96.3%] course of radiation; 5-fluorouracil-based chemotherapy [CT; 82%]; 18% did not receive CT). Rectal tumors were classified as cTNM (UICC TNM 8^{th} edition) I (1.2%), II (27.3%), III (71.5%) according to baseline staging information provided by pelvic magnetic resonance and chest/abdominal computed tomography imaging. An additional cohort of patients (n=73) with a complete/nearly complete response to nT (ycTNM 0-1), followed by a Watch-and-Wait strategy, was analyzed (Table 2). The median duration of follow-up for DFS of the cohort 1+2 was 45.4 months (IQR=25.7-65.6). Duration of follow-up of each cohort for DFS, TTR and OS with the number of events is provided in Table 3. The study was approved by an ethical review board of each center.

Clinical Outcomes

Patients were compared according to the degree of tumoral response to nT, using different tumor regression grade (TRG) scoring systems: i/ the Dworak classification (21) defined as complete (Dworak 4), near complete (Dworak 3), moderate (Dworak 2), minimal (Dworak 1) and no regression (Dworak 0), ii/ the neoadjuvant rectal (NAR) score classification (5), calculated using the equation [5pN-3(cT-pT)+12]\hat{ }2/9.61, and classified as low (<8), intermediate (8-16), and high (>16), iii/ the ypTNM stage, ie. the postsurgical pathological T and N evaluation, and iv/ downstaging of the tumor (4), defined as complete (ypT0N0), intermediate (ypT1-2N0), or weak/absent (ypT3-4 or N+). For patients who underwent surgery, the events were local, systemic recurrences and death from the date of surgery for disease-free survival (DFS), recurrences for time to recurrence (TTR), and death from any cause for overall survival (OS). All patients who were managed with the Watch-and-Wait strategy were considered to have clinical complete response (ycTNM0) and were offered a strict surveillance protocol.

Immunohistochemistry

Initial biopsies of all patients performed for diagnosis purpose were retrieved from all centers. Two formalin-fixed paraffin-embedded (FFPE) tumor tissue sections of 4 μm were processed for immunochemistry with antibodies against CD3+ (2GV6, 0.4 μg/mL; Ventana, Tucson, Ariz., USA) and CD8+ (C8/144B, 3 μg/mL; Dako, Glostrup, Denmark) according to the previously described protocol (17) revealed with the Ultraview Universal DAB IHC Detection Kit (Ventana, Tucson, Ariz., USA), and counterstained with Mayer's hematoxylin.

Biopsy-Based Immunoscore (IS_{B}) Determination

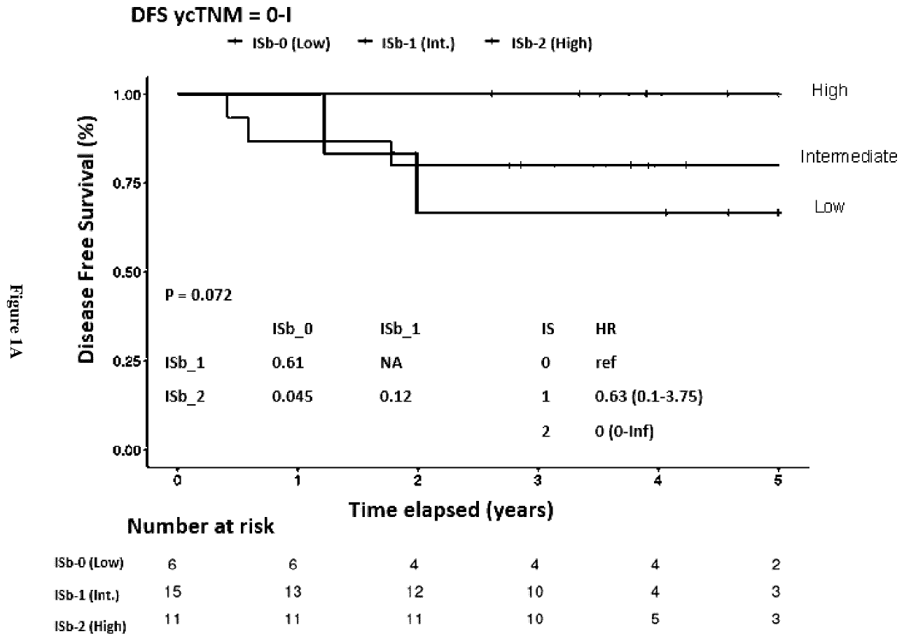

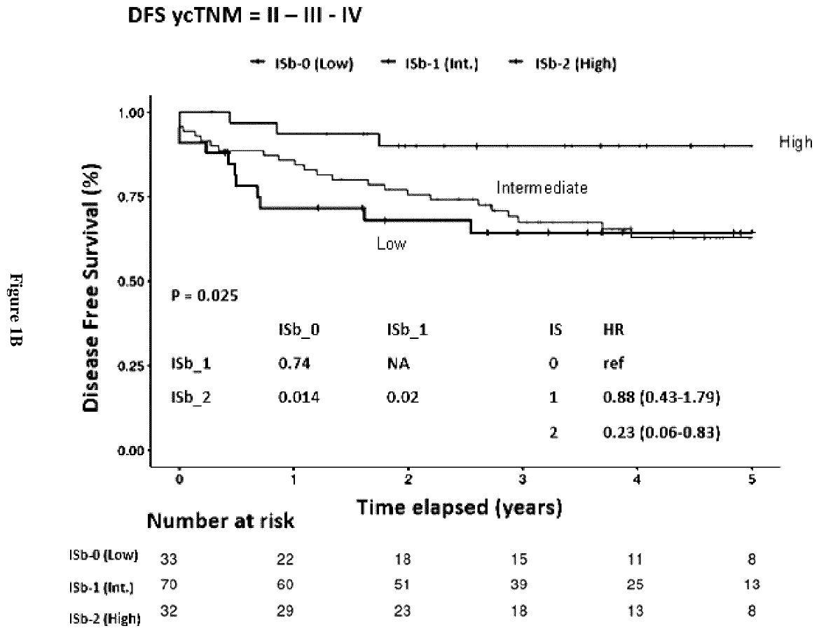

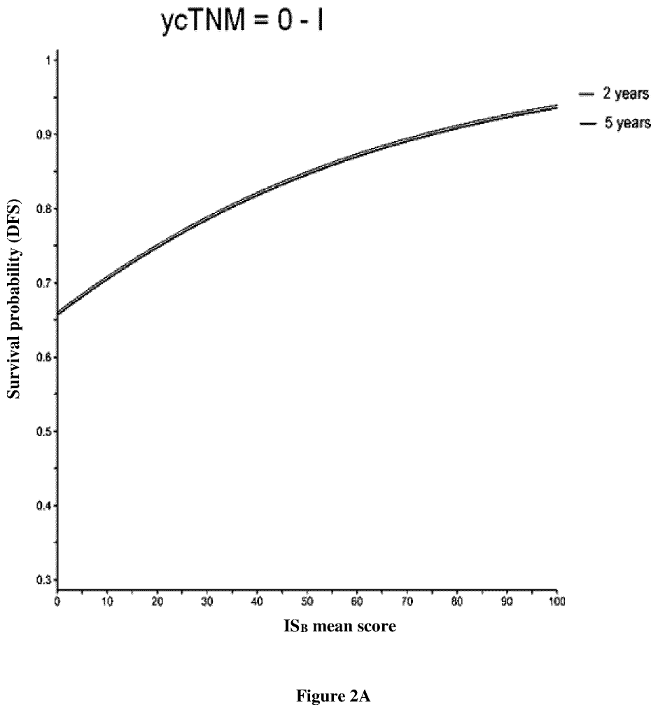

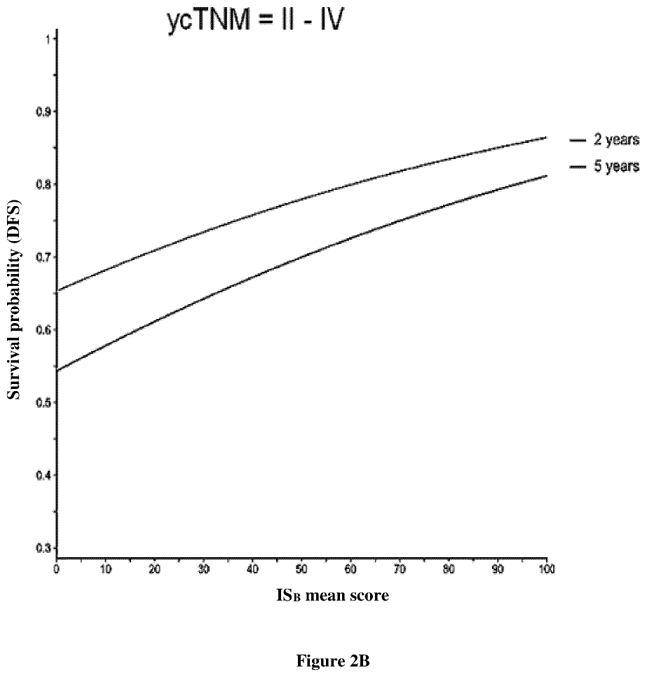

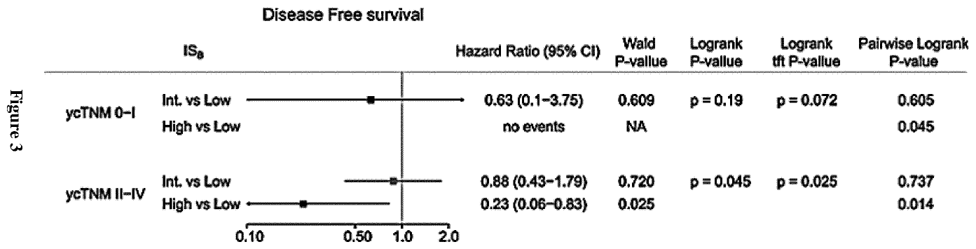

Digital images of stained tissue sections were obtained with a 20× magnification and a resolution of 0.45 μm/pixel (Nanozoomer H T, Hamamatsu, Japan). Delimitation of the tumoral component excluding normal tissue and low/high grade dysplasia-associated lesions was performed by an experienced pathologist (CL). The mean densities of CD3+ and CD8+ T cells in the tumor region were determined with a dedicated IS module of the Developer XD image analysis software (Definiens, Munich, Germany). The mean and distribution of the staining intensities were monitored providing an internal staining quality control. A final quality check was performed to remove nonspecific staining detected by the software. Determination of IS_{B} was directly derived from the methodology used to determine the Immunoscore (IS) in the international validation cohort of IS in colon cancers which have shown a strong inter-observer reproducibility (17). CD3+ and CD8+ T cells densities in the tumoral region of each patient were compared to that obtained for the whole cohort of patient and converted accordingly into percentile. Then, the mean of the two percentiles (CD3 and CD8) was translated into one of the three IS_{B} categories (FIG. 1B): IS_{B} Low (0-25%), IS_{B} Intermediate (>25-70%), and IS_{B} High (>70-100%). The IS_{B} determination was performed blinded to the study endpoint.

RNA Extraction and Transcriptomic Analysis by NanoString Technology

Total RNA from 20 μm FFPE tumor tissue sections from all patients for which both biopsies and the corresponding surgical specimen post-nT was available (cohort 1 and 2; n=62) and from colorectal cancer patients not treated with nT (n=13) was isolated using the RecoverAll™ Total Nucleic Acid Isolation Kit (Ambion ThermoFisher, Monza, Italy). Distribution of tumor extension T and N stages among patients with or without nT did not display any statistical difference. The quality and quantity of the isolated RNA was measured using Agilent RNA 6000 Nano kit (Agilent Technologies, Santa Clara, Calif.) and NanoDrop 2000 (ThermoFisher Scientific, Waltham, USA) and 100-400 ng RNA of each sample was processed using an in-house panel of 44 immune-related genes (Nanostring Technologies, Seattle, Wash., USA). Reporter-capture probe pairs were hybridized and the probe/target complexes were immobilized and counted on the nCounter analyzer. Background subtraction was applied to raw data and normalization based on the geometric mean of positive control and internal housekeeping genes (GUSB, SP2) was performed using the nSolver Analysis software, version 2.5.

Statistical Analysis and Data Visualization

Statistical analyses and data visualizations were performed using the R software version 3.5.1 with the add-on survival, survminer, ggpubr, ggplot2, rms and coin packages. The associations between IS_{B} and clinical characteristics were assessed through the chi-square or Fisher tests of independence. Association level between CD3+ and CD8+ cell densities was measured by Pearson's correlation coefficient r and related P value. Survival univariate analyses were performed using the log-rank test and the Cox proportional hazards model. Survival curves were estimated by the Kaplan-Meier method. The log-rank test for trend from the survminer package was performed to detect ordered differences in survival curves. Multivariate survival analyses were performed with Cox proportional hazards model to test the simultaneous influence of all covariates. The proportional hazards assumption (PHA) for each covariate was tested using the cox.zph function. The relative importance of each parameter to survival risk was assessed by the chi-square from Harrell's rms R package. The association between IS_{B} and nT ordinal response level was assessed using a unilateral linear-by-linear association test. The associations between nT response levels and CD3+, CD8+ T cells densities and gene intensities were assessed by Kendall's correlation test, T test, and Mann-Whitney U test. Wilcoxon test adjusted to control false discovery rate by using the Benjamini and Hochberg procedure was used to test treatment response level in transcriptional analysis. The ycTNM staging and IS_{B} were included in the proportional odds ordinal logistic regression model to predict good histopathologic response to nT. P values<0.05 were considered statistically significant. Principal component analysis (PCA) was performed with PCA and fviz_pca_ind functions from packages FactoMineR and factoextra. Linear weighted kappa was used to measure the agreement between resected tumors and biopsy samples in IS calculation.

Results:

Biopsy-Based Immunoscore (IS_{B}) Determination on the Rectal Cancer Diagnostic Tissues

CD3+ lymphocytes and cytotoxic CD8+ cells were assessed on initial tumor biopsies performed for diagnosis purpose of LARC (n=322) treated by nT. The immunostaining intensity was monitored to ensure a valid detection and counting of stained cells with the image analysis software (not shown). Seven patients were excluded after biomarker quality control (2.8%), and 4 patients were excluded after clinical data quality control (1.2%). The median density of CD3+ and CD8+ T cells in the tumor were 1363 cells/mm² and 274 cells/mm², respectively (data not shown). The CD3+/CD8+ T cells ratio was highly variable among patients, with a coefficient of determination (r²) between both markers of 0.58 (data not shown). IS_{B} was derived from the CD3+ and CD8+ T cells densities (data not shown). CD3 and CD8 densities in the tumor were converted into percentiles referring to the densities observed in all patients. IS_{B} mean percentile of CD3 and CD8 was calculated for each biopsy (IS_{B} mean score). No difference for the mean score was observed between the two cohorts (data not shown). After converting the mean score into IS_{B} scoring system, overall 22.7%, 52.5%, and 24.8% of patients had IS_{B} Low, Intermediate and High, respectively. Of note, IS_{B} Intermediate category was more represented in the cohort 2 (61.9%), as compared to cohort 1 (43.5%).

Biopsy-Based Immunoscore (IS_{B}) Associated Prognostic Value

Distribution analysis of IS_{B} did not display any association with age, sex or tumor location (Table 1). The magnitude and reproducibility of the IS_{B} prognostic performance were tested in two independent cohorts. In cohort 1 (n₁=131), a significant difference in DFS between patients stratified by IS_{B} was observed (P test for trend [P_{tft}]=0.012; HR_{[High versus Low]}=0.21 (95% CI 0.06-0.78)). Patients with IS_{B} High were at low-risk of relapse, with the 5-year DFS of 91.1% (95% CI 82.0-100) versus 65.8% (95% CI 49.8-86.9) in patients with IS_{B} Low. These results were confirmed in second independent cohort (n₂=118; P_{tft}=0.021; HR_{[High versus Low]}=0.25, 95% CI 0.07-0.86). Identical results were obtained when removing the 3 patients with UICC-TNM stage I tumors (data not shown). In pooled analysis (n=249), a significant difference between patient's groups stratified by IS_{B} was evidenced by univariate analysis (data not shown) and illustrated by Kaplan-Meier curves for TTR (P<0.001), DFS (P<0.005), and OS (P=0.04; data not shown).

Biopsy-Based Immunoscore (IS_{B}) and Response to Neoadjuvant Treatment

We investigated if the prognostic value associated to IS_{B} was at least partly a consequence of a relationship between IS_{B} and the quality of the nT response. The quality of response to nT is assessed 6 to 8 weeks after nT by imaging (ycTNM) and microscopic examination of the resected tumor, by the Dworak classification, a tumor regression grading system, ypTNM, downstaging and the neoadjuvant rectal (NAR) score. In our cohorts (n=249 patients), high CD3+ and CD8+ T cells densities were significantly associated with a good response to nT evaluated by both Dworak classification and ypTNM staging (all P<0.005; data not shown). The mean of CD3+ and CD8+ percentiles (IS_{B} mean score) was correlated with the NAR score, Dworak classification, and ypTNM staging (data not shown). The IS_{B} level and distribution was positively correlated with tumor response to nT (data not shown). IS_{B} High patients were not found in the non-responder Dworak 0 group, and 52.9% of patients with undetectable tumor cells (ie. the Dworak 4 group) were IS_{B} High (P=0.0006). The same correlation was observed with the ypTNM, tumor downstaging, and NAR (data not shown). Good responders to nT were six times more frequent in the IS_{B} High group than in the IS_{B} Low group according to the NAR scoring system (data not shown). Immune consequences of nT were then investigated on post-nT tumor samples (Dworak 0-4; n=62) by analyzing 44 immune-related genes (data not shown). Gene expression levels were highly variable among patients (data not shown). Unsupervised hierarchical clustering showed that 31.7% (n=19) of patients presented with signs of local immune activation after nT (data not shown). The immune activation status after nT was positively correlated with the densities of CD3+ and CD8+ T cells (i.e. IS_{B}) before treatment (data not shown). Non-responder tumors (Dworak 0-1) presented similarly low level of immune-related genes expression compared to tumors not treated by nT (data not shown). Patients with a partial/complete response to neoadjuvant treatment had a significantly higher expression of genes associated with adaptive immunity (CD3D, CD3E, CD3Z, CD8A), Th1 orientation (TBX21 Tbet, STAT4), activation (CD69), cytotoxicity (GZMA, GZMH, GZMK, PRF1), immune checkpoints (CTLA-4, LAG3), and chemokines (CCL2, CCL5, CX3CL1), as compared to patients non-responders to nT (data not shown). This suggests a link between the quality of the natural adaptive cytotoxic immune response (IS_{B}), the presence of a post-nT immune activation and the degree of response to nT. Gene expression data analysis through a Principal Component Analysis (PCA) visualization further reinforced the putative link existing between the response to nT and the immune environment by showing distinct patterns of gene expression depending on the degree of response to nT (data not shown). “The combination of the second and third dimension was the most accurate to discriminate responders/non-responders.

Biopsy-Adapted Immunoscore (IS_{B})—a Biomarker to Optimize Patient Care

We investigated whether the IS_{B} could provide valuable prognostic information when combined with clinic and pathologic criteria available (i) before nT (i.e. initial imaging, cTNM (UICC TNM 8^{th} edition)), (ii) after nT (ie. imaging post-nT, ycTNM) and (iii) after surgery (pathologic examination, ypTNM). In Cox multivariate analysis, IS_{B} was a stronger predictive marker of DFS than other clinicopathological parameters including cTNM (IS_{B} High versus IS_{B} Low: HR=0.2, P<0.001) and ycTNM (IS_{B} High versus IS_{B} Low: HR=0.25, P=0.039). IS_{B} further remained a significant independent parameter associated with DFS when combined to ypTNM (Table 4). It is known that the accuracy of the complete response post-nT defined by imaging is imperfect. Thus, only 25 to 50% of clinical complete responders have no residual tumor (i.e. complete histologic response) (22-24). IS_{B} combined to imaging post-nT (ycTNM) increased the accuracy of prediction of histological good responders (ypTNM 0-I) as compared to ycTNM alone. Three out of 32 patients with good response to nT (ycTNM=0-I, n=32) experienced distant relapses, and no local relapse were observed. Importantly, no relapse was observed in IS_{B} High patients (FIGS. 1A,B, 2A,B and 3). Thus, IS_{B} could help to select patients who could achieve a very favorable outcome and be eligible to a Watch and Wait strategy.

IS_{B} in Patients Managed with Watch-and-Wait Strategy

In a series of patient treated by Watch-and-Wait strategy (n=73), we retrieved the initial diagnostic biopsies to evaluate the IS_{B} and the associated clinical outcome. Overall, 23%, 51%, and 26% were classified as IS_{B} High, IS_{B} Intermediate, and IS_{B} Low, respectively. Time to relapse was significantly different among patients stratified for IS_{B} (P_{[High versus Low]}=0.025; data not shown). No evidence of relapse was noticed during the follow-up period in IS_{B} High patients. Under the Cox proportional hazards regression model, the 5-year probability of survival without recurrence ranged from 46% to 89% according to IS_{B} Mean Score (data not shown). In Cox multivariable analysis, IS_{B} was related to the patient's TTR, independent of age, tumor location, and the cTNM classification (UICC TNM 8^{th} edition) (P_{[High versus Low]}=0.04; data not shown).

### DISCUSSION

This work highlights the links between (i) the quality of natural intra-tumor immunity evaluated by the IS_{B} (ii) the intensity of in situ immune reaction post-nT (iii) the extend of the tumor regression post-nT and (iv) the clinical impact in terms of prevention of tumor recurrence and survival. From a clinical point of view, IS_{B} provides a reliable estimate of both the quality of response after nT and of the risk of recurrence and death in LARC patients. IS_{B} combined with imaging, could further identify patients with a complete clinical response whom can benefit from a close surveillance strategy post-nT, thus avoiding a disabling and useless rectal amputation surgery.

IS_{B} can be performed on routine diagnosis biopsies without any additional medical procedure. The rigorous and standardized quantification of immune cell infiltrates was achieved as for the IS colon study (17).

In the current study, IS_{B} was positively and significantly correlated with tumor response to nT. This observation is consistent with our previous preliminary result (18) and with studies using an optical semi-quantitative evaluation of immune cell infiltrates (19,20,25). In the IS_{B} Low group (22.7% of the cohort), only 5% of patients experienced complete response (Low NAR score), suggesting that an optimization or modification of nT such as adjunctive therapies (26), immunotherapy (27), or drug repositioning may provide greater benefits for these patients in order to achieve a better response. We evidenced an association between signs of in situ cytotoxic adaptive immune response and inflammatory interferon type I-associated molecules production post-nT and the response to treatment. Type I IFNs play a key role in antitumoral immunity by promoting the maturation and presentation capacity of dendritic cells and their migration to lymph nodes (28). This immune state was influenced by the quality and the intensity of the natural immune response preexisting before nT. IS_{B} High could not only favor nT-dependent tumor cell death, but also promote the presence of resident immune components that could be essential to avoid local recurrences in organ preservative strategies such as Watch-and-Wait. Of note, few IS_{B} high patients did not achieve a good response, highlighting that treatment resistance is also guided by independent tumor intrinsic factors (29) or the presence of a suppressive microenvironment (30). Neoadjuvant treatment with development of clinical complete response post-nT has raised the possibility of organ-preserving strategies, because radical resection of the rectum results in functional outcomes, immediate morbidity, and even mortality rates (31). However, imaging after nCRT (ycTNM) has low accuracy in predicting pathologic complete response due to over or under-staging (32). Importantly, no relapse was observed in good responders with IS_{B} High patients. In addition, IS_{B} increased the accuracy prediction for very good responders (ypTNM 0-I) evaluated by imaging and identified a subgroup of patients treated with an organ-preserving strategy (Watch-and-Wait) with a very favorable outcome. No biomarker is currently available to help selection of good responders eligible to Watch-and-Wait strategy (9). These results may have significant implication in selecting potential candidates for organ-preservation including patients with IS_{B} High and complete clinical response to nT, but also those with a delayed complete clinical response (i.e. “nearly-complete responders”) that are presently classified as incomplete responders (33). This study has some limitations. The immune densities associated with predefined cut points (i.e. 25^{th} and 70^{th} percentile) are closely linked to the clinical characteristics of the studied cohort. The densities used as cut point are relevant to LARC patients treated by nT before surgery. In addition, assessment of IS_{B} was performed on initial biopsies; this implies analysis of only a small fraction of the tumor (10-15% of the surface of cut from a tumor block available after TME) and no analysis of the invasive margin not present on biopsies. In order to evaluate the correspondence of IS_{B} and IS in resected tumor, we analyzed 33 colon cancer biopsies and their associated resected tumor, we found a partial correlation between these two specimens (data not shown, kappa=0.45, p=0.0004). All discrepancies were only observed between 2 consecutive categories of IS. Despite this limited surface analysis and the absence of invasive margin, the prognostic value of the IS_{B} was retained suggesting the accuracy of the immune evaluation on initial diagnostic biopsy when the surgical piece is unavailable or is impossible to analyze due to architectural changes secondary to the neoadjuvant treatment. In addition, performing IS on post-operative specimen would not allow an assessment of its predictive value of the response to nT. Furthermore, due to the deep histological modifications after nT (no clear delimitation of the tumor and its invasive margin) an IS on post nT specimen is not feasible. The study was performed on patients who came from different countries and received standard-of-care treatment in real-life clinical practice. Despite the size of the specimen and the multiple types of patient care, the strong and constant prognosis value associated with IS_{B}, highlight the robustness of the test, and its generalizability. Prognostic parameters such as mismatch repair, KRAS, and BRAF status not available in our study, were not included in multivariate analysis with IS scoring system. However, MSI+ cases are rare in rectal cancer (<5%) (34), and we recently evidenced that IS was an independent prognostic parameter for survival when associated with MSI, KRAS and BRAF status in colonic cancers (35). Most of the rectal cancer included in this study was adenocarcinomas. A sub-analysis by histologic subtypes could not be performed due to the large multicentric character of the cohorts studied, with heterogeneous level of histopathological description and the obvious small effective of mucinous carcinomas, signet ring cell carcinoma, or tumor budding to address their relative prognostic impact with enough power. This study emphasizes the importance of the initial diagnostic biopsies, often done in private practices, and not easily available in some cases. Rectal cancer patients would benefit from a close partnership between private pathology practices, clinics, and teaching hospitals in order to initially assess their immune status (IS_{B}). This material could become in the near future essential and be part of the personal medical file of rectal cancer patient as it is the sole material available before any neoadjuvant treatment. IS_{B} may facilitate a personalized multimodal treatment of rectal cancer particularly in patients with IS_{B} High tumors at baseline and with signs of tumor regression by imaging. These patients should benefit the most from the conservative strategy and in turns preserve their quality of life.

In conclusion, our results indicate that IS_{B} could be used (i) to predict tumor response after nT (ii) to re-stage local disease after nT, and (iii) to predict clinical outcome. This method may facilitate a personalized multimodal treatment of rectal cancer particularly in patients with IS_{B} High tumors at baseline and with signs of tumor regression by imaging. These patients should benefit the most from the conservative strategy and in turns preserve their quality of life. IS_{B} is yet to be validated on larger Watch-and-Wait cohorts both retrospectively and prospectively. Such validations are planned in international collaboration studies using the International Watch-and-Wait Database and in the OPERA ongoing clinical trial (NCT02505750).

TABLE 1
Clinical characteristics of patients who underwent total mesorectal excision according to IS_{B}

| | Cohort 1 | | | | | | | Cohort 2 | | | | | | |
|---|---|---|---|---|---|---|---|---|---|---|---|---|---|---|
| | IS_{B} Low | | IS_{B} Int | | IS_{B} High | | | IS_{B} Low | | IS_{B} Int | | IS_{B} High | | |
| Characteristics | No. (%) | | No. (%) | | No. (%) | | P | No. (%) | | No. (%) | | No. (%) | | P |
| No. of patients | | | | | | | | | | | | | | |
| Age | | | | | | | 0.58* | | | | | | | 0.12* |
| ≤65 years | 12 | (40) | 24 | (44.4) | 21 | (52.5) | | 15 | (60) | 28 | (38.4) | 11 | (55) | |
| >65 years | 18 | (60) | 30 | (55.6) | 19 | (47.5) | | 10 | (40) | 45 | (61.6) | 61.6 | (9) | |
| Gender | | | | | | | 0.79* | | | | | | | 0.10* |
| Male | 18 | (60) | 36 | (66.7) | 27 | (67.5) | | 18 | (72) | 51 | (69.9) | 9 | (45) | |
| Female | 12 | (40) | 18 | (33.3) | 13 | (32.5) | | 7 | (28) | 22 | (30.1) | 11 | (55) | |
| Tumor location | | | | | | | 0.92* | | | | | | | 0.59* |
| Inferior | 15 | (50) | 28 | (51.9) | 23 | (57.5) | | 14 | (56) | 33 | (45.8) | 10 | (50) | |
| Middle | 10 | (33.3) | 18 | (33.3) | 10 | (25) | | 10 | (40) | 34 | (47.2) | 7 | (35) | |
| Superior | 5 | (16.7) | 8 | (14.8) | 7 | (17.5) | | 1 | (4) | 5 | (6.9) | 3 | (15) | |
| cTMM stage | | | | | | | 0.08† | | | | | | | 0.03† |
| I | 1 | (3.3) | 1 | (1.9) | — | | | — | | — | | 1 | (5) | |
| II | 14 | (46.7) | 21 | (38.9) | 9 | (22.5) | | 5 | (20) | 9 | (12.3) | 7 | (35) | |
| III | 15 | (50) | 32 | (59.3) | 21 | (77.5) | | 20 | (80) | 64 | (87.7) | 12 | (60) | |
| ycTNM stage | | | | | | | 0.36† | | | | | | | 0.04† |
| 0 | 1 | (4.3) | — | | 3 | (8.3) | | — | | — | | — | | |
| I | 2 | (8.7) | 9 | (22.5) | 4 | (11.1) | | 3 | (16.7) | 6 | (12.8) | 4 | (57.1) | |
| II | 15 | (65.2) | 18 | (45) | 19 | (52.8) | | 3 | (16.7) | 8 | (17) | 1 | (14.3) | |
| III | 5 | (21.7) | 12 | (30) | 10 | (27.8) | | 8 | (44.4) | 31 | (66) | 2 | (28.6) | |
| IV | — | | 1 | (2.5) | — | | | 4 | (22.2) | 2 | (4.3) | — | | |
| ypTNM stage | | | | | | | 0.69† | | | | | | | 0.05† |
| 0 | 2 | (67) | 5 | (9.4) | 5 | (12.5) | | — | | 1 | (1.4) | 3 | (15) | |
| I | 5 | (16.7) | 15 | (28.3) | 13 | (32.5) | | 4 | (17.4) | 20 | (28.2) | 8 | (40) | |
| II | 11 | (36.7) | 17 | (32.1) | 14 | (35) | | 9 | (39.1) | 23 | (32.4) | 5 | (25) | |
| III | 11 | (36.7) | 14 | (26.4) | 8 | (20) | | 6 | (26.1) | 24 | (33.8) | 4 | (20) | |
| IV | 1 | (3.3) | 2 | (3.8) | — | | | 4 | (17.4) | 3 | (4.2) | — | | |
| Dworak | | | | | | | 0.36† | | | | | | | 0.05† |
| classfication | | | | | | | | | | | | | | |
| 0 | — | | 3 | (6) | — | | | 2 | (10) | 6 | (10.2) | — | | |
| 1 | 7 | (25.9) | 7 | (14) | 4 | (10.5) | | 9 | (45) | 21 | (35.6) | 2 | (20) | |
| 2 | 14 | (51.9) | 19 | (38) | 19 | (50) | | 6 | (30) | 21 | (35.6) | 3 | (30) | |
| 3 | 4 | (14.8) | 16 | (32) | 10 | (26.3) | | 3 | (15) | 10 | (16.9) | 1 | (10) | |

| | | | | | | | | | | | |
|---|---|---|---|---|---|---|---|---|---|---|---|
| 4 | 2 | (7.4) | 5 | (10) | 5 | (13.2) | — | 1 | (1.7) | 4 | (40) |

NOTE
*Chi square P value for independence between clinical variables and IS_{B} level
†Fisher's test P value for independence between clinical variables and IS_{B} level
— not applicable

TABLE 2
Clinical characteristics of patients who underwent the Watch-and-Wait
strategy according to IS_{B}.

| | Overall | | IS_{B} Low | | IS_{B} Int | | IS_{B} High | | |
|---|---|---|---|---|---|---|---|---|---|
| Characteristics | No. (%) | | No. (%) | | No. (%) | | No. (%) | | P |
| No. of patients | 73 | (100) | 19 | (26) | 37 | (50.7) | 17 | (23.3) | — |
| Age | | | | | | | | | 0.28* |
| ≤65 years | 32 | (43.8) | 6 | (31.6) | 16 | (43.2) | 10 | (58.8) | |
| >65 years | 41 | (56.2) | 13 | (68.4) | 21 | (56.8) | 7 | (41.2) | |
| Gender | | | | | | | | | 0.32* |
| Male | 40 | (54.8) | 10 | (52.6) | 23 | (62.2) | 7 | (41.2) | |
| Female | 33 | (45.2) | 9 | (47.4) | 14 | (37.8) | 10 | (58.8) | |
| Tumor location | | | | | | | | | 0.04† |
| Inferior | 43 | (81.1) | 15 | (100) | 20 | (80) | 8 | (61.5) | |
| Middle | 9 | (17) | 0 | (0) | 5 | (20) | 4 | (30.8) | |
| Superior | 1 | (1.9) | 0 | (0) | 0 | (0) | 1 | (7.7) | |
| cTNM stage | | | | | | | | | 0.02† |
| I | 9 | (17.3) | 2 | (14.3) | 7 | (28) | 0 | (0) | |
| II | 10 | (192) | 6 | (42.9) | 2 | (8) | 2 | (15.4) | |
| III | 33 | (63.5) | 6 | (42.9) | 16 | (64) | 11 | (84.6) | |

*Chi square P value for independence between clinical variables and IS_{B} level
†Fisher’s test P value for independence between clinical variables and IS_{B} level
— Not applicable

TABLE 3
Median survival [+/− IQR] and event numbers

| | | | | | | [symbol] elapse | Relapse or |
|---|---|---|---|---|---|---|---|
| | OS (months) | | Death Nb. | TTR/DFS (months) | | N [symbol] | Death Nb. |
| All | 45.4 | (25.7-65.5) | 55 | 38.5 | (16.4-58) | 60 | 79 |
| cohort 1 | 55 | (32.3-91.3) | 22 | 46.4 | (23.7-81.7) | 23 | 30 |
| cohort 2 | 36.9 | (18.6-48.3) | 33 | 32.5 | (10.4-47.7) | 37 | 49 |
| W&W | 37.1 | (24.2-59.2) | 15 | 31.5 | (20.3-45.6) | 14 | 22 |

[symbol]

indicates data missing or illegible when filed

TABLE 4
Multivariate Cox models for disease-free survival according to biopsy-adapted Immunoscore (IS_{B}) combined with available clinical parameters

| | Before neoadjuvant treatment | | | | After neoadjuvant treatment | | | | After surgery | | | |
|---|---|---|---|---|---|---|---|---|---|---|---|---|
| | PHA | HR | | P | PHA | HR | | P | PHA | HR | | P |
| Characteristics | test | (95% CI) | | value* | test | (95% CI) | | value* | test | (95% CI) | | value [symbol] |
| Age | | | | | | | | | | | | |
| Under vs Over 65 years | 0.922 | 1.38 | (0.85-2.24) | 0.19 | 0.432 | 1.41 | (0.69-2.9) | 0.346 | 0.636 | 1.46 | (0.86-2.48) | 0.159 [symbol] |
| Tumor location | | | | | | | | | | | | |
| Middle vs Inferior | 0.43 | 1.1 | (0.67-1.8) | 0.72 | 0.688 | 0.9 | (0.45-1.81) | 0.766 | 0.894 | 0.83 | (0.47-1.46) | 0.527 [symbol] |
| Superior vs Inferior | 0.5 | 0.66 | (0.26-1.69) | 0.39 | 0.583 | 0.3 | (0.04-2.24) | 0.24 | 0.212 | 0.74 | (0.29-1.9) | 0.529 [symbol] |
| Sex | | | | | | | | | | | | |
| Male vs Female | 0.06 | 1.54 | (0.9-2.63) | 0.118 | 0.131 | 1.87 | (0.8-4.37) | 0.148 | 0.033 | 1.38 | (0.76-2.49) | 0.291 [symbol] |
| Immunoscore (IS_{B}) | | | | | | | | | | | | |
| Intermediate vs Low | 0.476 | 0.65 | (0.38-1.1) | 0.111 | 0.906 | 0.76 | (0.33-1.73) | 0.508 | 0.708 | 0.93 | (0.5-1.71) | 0.807 [symbol] |
| High vs Low | 0.83 | 0.2 | (0.08-0.49) | <0.001 | 0.834 | 0.25 | (0.07-0.93) | 0.039 | 0.209 | 0.34 | (0.13-0.89) | 0.028 [symbol] |
| cTNM stage | | | | | | | | | | | | |
| III vs I-II | 0.59 | 1.18 | (0.68-2.04) | 0.56 | — | — | | — | — | — | | — |
| ycTNM stage | | | | | | | | | | | | |
| 0 vs III | — | — | | — | — | — | | — | — | — | | — |
| I vs III | — | — | | — | 0.916 | 0.62 | (0.23-1.69) | 0.349 | — | — | | — |
| II vs III | — | — | | — | 0.257 | 0.48 | (0.22-1.08) | 0.076 | — | — | | — |
| ypTNM stage | | | | | | | | | | | | |

| | | | | | | | | | | |
|---|---|---|---|---|---|---|---|---|---|---|
| 0 vs III | — | — | — | — | — | — | 0.208 | 0.14 | (0.02-1.01) | 0.051 [symbol] |
| I vs III | — | — | — | — | — | — | 0.071 | 0.2 | (0.09-0.45) | <0.00 [symbol] |
| II vs III | — | — | — | — | — | — | 0.207 | 0.51 | (0.29-0.89) | 0.018 [symbol] |

*The significance of the multivariate Cox regression model was evaluated with the Wald test
— not applicable
IS, Immunoscore;
PHA, proportional hazards assumption;
HR, Hazard ratio [symbol]
indicates data missing or illegible when filed

### REFERENCES

Throughout this application, various references describe the state of the art to which this invention pertains. The disclosures of these references are hereby incorporated by reference into the present disclosure.
1. Araghi M, Soerjomataram I, Bardot A, Ferlay J, Cabasag C J, Morrison D S, et al. Changes in colorectal cancer incidence in seven high-income countries: a population-based study. The Lancet Gastroenterology & Hepatology. 2019; 4:511-8.
2. Nielsen L B J, Wille-Jørgensen P. National and international guidelines for rectal cancer. Colorectal Disease. 2014; 16:854-65.
3. Glynne-Jones R, Wyrwicz L, Tiret E, Brown G, Rödel C, Cervantes A, et al. Rectal cancer: ESMO Clinical Practice Guidelines for diagnosis, treatment and follow-up†. Annals of Oncology. 2017; 28:iv22-40.
4. Park I J, You Y N, Agarwal A, Skibber J M, Rodriguez-Bigas M A, Eng C, et al. Neoadjuvant Treatment Response As an Early Response Indicator for Patients With Rectal Cancer. J Clin Oncol. 2012; 30:1770-6.
5. George T J, Allegra C J, Yothers G. Neoadjuvant Rectal (NAR) Score: a New Surrogate Endpoint in Rectal Cancer Clinical Trials. Curr Colorectal Cancer Rep. 2015; 11:275-80.
6. Rödel C, Martus P, Papadoupolos T, Füzesi L, Klimpfinger M, Fietkau R, et al. Prognostic Significance of Tumor Regression After Preoperative Chemoradiotherapy for Rectal Cancer. Journal of Clinical Oncology. 2005; 23:8688-96.
7. Habr-Gama A, Perez R O, Nadalin W, Sabbaga J, Ribeiro U, Silva e Sousa A H, et al. Operative Versus Nonoperative Treatment for Stage 0 Distal Rectal Cancer Following Chemoradiation Therapy: Long-term Results. Transactions of the. Meeting of the American Surgical Association. 2004; CXXII:309-16.
8. Habr-Gama A. Assessment and management of the complete clinical response of rectal cancer to chemoradiotherapy. Colorectal Disease. 2006; 8:21-24.
9. van der Valk M J M, Hilling D E, Bastiaannet E, Meershoek-Klein Kranenbarg E, Beets G L, Figueiredo N L, et al. Long-term outcomes of clinical complete responders after neoadjuvant treatment for rectal cancer in the International Watch & Wait Database (IWWD): an international multicentre registry study. The Lancet. 2018; 391:2537-45.
10. Demaria S, Ng B, Devitt M L, Babb J S, Kawashima N, Liebes L, et al. Ionizing radiation inhibition of distant untreated tumors (abscopal effect) is immune mediated. International Journal of Radiation Oncology•Biology•Physics. 2004; 58:862-70.
11. Rodríguez-Ruiz M E, Vanpouille-Box C, Melero I, Formenti S C, Demaria S. Immunological Mechanisms Responsible for Radiation-Induced Abscopal Effect. Trends in Immunology. 2018; 39:644-55.
12. Chajon E, Castelli J, Marsiglia H, Crevoisier R D. The synergistic effect of radiotherapy and immunotherapy: A promising but not simple partnership. Critical Reviews in Oncology/Hematology. 2017; 111:124-32.
13. Fridman W H, Pagès F, Sautès-Fridman C, Galon J. The immune contexture in human tumours: impact on clinical outcome. Nat Rev Cancer. 2012; 12:298-306.
14. Pagès F, Berger A, Camus M, Sanchez-Cabo F, Costes A, Molidor R, et al. Effector Memory T Cells, Early Metastasis, and Survival in Colorectal Cancer. New England Journal of Medicine. 2005; 353:2654-66.
15. Galon J. Type, Density, and Location of Immune Cells Within Human Colorectal Tumors Predict Clinical Outcome. Science. 2006; 313:1960-4.
16. Shinde V, Burke K E, Chakravarty A, Fleming M, McDonald A A, Berger A, et al. Applications of Pathology-Assisted Image Analysis of Immunohistochemistry-Based Biomarkers in Oncology. Vet Pathol. 2014; 51:292-303.
17. Pagès F, Mlecnik B, Marliot F, Bindea G, Ou F-S, Bifulco C, et al. International validation of the consensus Immunoscore for the classification of colon cancer: a prognostic and accuracy study. The Lancet. 2018; 391:2128-39.
18. Anitei M-G, Zeitoun G, Mlecnik B, Marliot F, Haicheur N, Todosi A-M, et al. Prognostic and Predictive Values of the Immunoscore in Patients with Rectal Cancer. Clinical Cancer Research. 2014; 20:1891-9.
19. Yasuda K, Nirei T, Sunami E, Nagawa H, Kitayama J. Density of CD4(+) and CD8(+) T lymphocytes in biopsy samples can be a predictor of pathological response to chemoradiotherapy (CRT) for rectal cancer. Radiat Oncol. 2011; 6:49.
20. Shinto E, Hase K, Hashiguchi Y, Sekizawa A, Ueno H, Shikina A, et al. CD8+ and FOXP3+ Tumor-Infiltrating T Cells Before and After Chemoradiotherapy for Rectal Cancer. Ann Surg Oncol. 2014; 21:414-21.
21. Dworak O, Keilholz L, Hoffmann A. Pathological features of rectal cancer after preoperative radiochemotherapy. International journal of colorectal disease. 1997; 12:19-23.
22. Hiotis S P, Weber S M, Cohen A M, Minsky B D, Paty P B, Guillem J G, et al. Assessing the predictive value of clinical complete response to neoadjuvant therapy for rectal cancer: an analysis of 488 patients1 1No competing interests declared. Journal of the American College of Surgeons. 2002; 194:131-5.
23. Rullier E, Rouanet P, Tuech J-J, Valverde A, Lelong B, Rivoire M, et al. Organ preservation for rectal cancer (GRECCAR 2): a prospective, randomised, open-label, multicentre, phase 3 trial. The Lancet. 2017; 390:469-79.
24. The 2017 European Society of Coloproctology (ESCP) collaborating group. Evaluating the incidence of pathological complete response in current international rectal cancer practice: the barriers to widespread safe deferral of surgery. Colorectal Dis. 2018; 20:58-68.
25. Matsutani S, Shibutani M, Maeda K, Nagahara H, Fukuoka T, Nakao S, et al. Significance of tumor-infiltrating lymphocytes before and after neoadjuvant therapy for rectal cancer. Cancer Science. 2018; 109:966-79.
26. Gash K J, Chambers A C, Cotton D E, Williams A C, Thomas M G. Potentiating the effects of radiotherapy in rectal cancer: the role of aspirin, statins and metformin as adjuncts to therapy. Br J Cancer. 2017; 117:210-9.
27. Ngwa W, Irabor O C, Schoenfeld J D, Hesser J, Demaria S, Formenti S C. Using immunotherapy to boost the abscopal effect. Nature Reviews Cancer. 2018; 18:313-22.
28. Zitvogel L, Galluzzi L, Kepp O, Smyth M J, Kroemer G. Type I interferons in anticancer immunity. Nat Rev Immunol. 2015; 15:405-14.
29. Makena M R, Ranjan A, Thirumala V, Reddy A P. Cancer stem cells: Road to therapeutic resistance and strategies to overcome resistance. Biochim Biophys Acta Mol Basis Dis. 2018; 165339.
30. Barker H E, Paget J T E, Khan A A, Harrington K J. The tumour microenvironment after radiotherapy: mechanisms of resistance and recurrence. Nat Rev Cancer. 2015; 15:409-25.
31. Smith F M, Rao C, Oliva Perez R, Bujko K, Athanasiou T, Habr-Gama A, et al. Avoiding radical surgery improves early survival in elderly patients with rectal cancer, demonstrating complete clinical response after neoadjuvant therapy: results of a decision-analytic model. Dis Colon Rectum. 2015; 58:159-71.
32. Sclafani F, Brown G, Cunningham D, Wotherspoon A, Mendes L S T, Balyasnikova S, et al. Comparison between MRI and pathology in the assessment of tumour regression grade in rectal cancer. British Journal of Cancer. 2017; 117:1478-85.
33. Hupkens B J P, Maas M, Martens M H, van der Sande M E, Lambregts D M J, Breukink S O, et al. Organ Preservation in Rectal Cancer After Chemoradiation: Should We Extend the Observation Period in Patients with a Clinical Near-Complete Response? Ann Surg Oncol. 2018; 25:197-203.
34. Nilbert M, Planck M, Fernebro E, Borg Å, Johnson A. Microsatellite instability is rare in rectal carcinomas and signifies hereditary cancer. European Journal of Cancer. 1999; 35:942-5.
35. Marliot F, Chen X, Kirilovsky A, Sbarrato T, Sissy C E, Batista L, et al. Analytical validation of the Immunoscore and its associated prognostic value in patients with colon cancer. J Immunother Cancer. BMJ Specialist Journals; 2020; 8:e000272.

1. A method of predicting the risk of recurrence and/or death of a patient suffering from a solid cancer after preoperative adjuvant therapy and treating the patient comprising
determining
i) an arithmetic mean value of percentile of an immune response of the patient before the preoperative adjuvant therapy, and
ii) a clinical response determined after the preoperative adjuvant therapy;
treating, by radical surgery and/or adjuvant therapy, the subject determined to have
iii) an arithmetic mean value of percentile of an immune response that is lower than a predetermined reference arithmetic mean value of percentile, and
iv) a partial clinical response or no clinical response to the preoperative adjuvant therapy.

2. The method of claim 1 wherein the patient suffers from a primary cancer or from a metastatic cancer.

3. The method of claim 1 wherein the patient suffers from a locally advanced cancer.

4. The method of claim 1 wherein the patient suffers from a locally advanced rectal cancer.

5. The method of claim 1 wherein the preoperative adjuvant therapy comprises radiotherapy, chemotherapy, a targeted therapy, a hormone therapy, an immunotherapy or a combination thereof.

6. The method of claim 1 wherein the preoperative adjuvant therapy comprises a combination of radiotherapy and chemotherapy.

7. The method of claim 1 wherein the immune response is assessed by quantifying one or more immune markers determined in a biopsy tumor sample obtained from the patient before the preoperative adjuvant chemotherapy.

8. The method of claim 7 wherein the one or more immune markers comprise the density of CD3+ cells, the density of CD8+ cells, the density of CD45RO+ cells, the density of GZM-B+ cells, the density of CD103+ cells and/or the density of B cells.

9. The method of claim 7 wherein the one or more immune markers comprise the density of CD3+ cells and the density of CD8+ cells, the density of CD3+ cells and the density of CD45RO+ cells, the density of CD3+ cells the density of GZM-B+ cells, the density of CD8+ cells and the density of CD45RO+ cells, the density of CD8+ cells and the density of GZM-B+ cells; the density of CD45RO+ cells and the density of GZM-B+ cells or the density of CD3+ cells and the density of CD103+ cells.

10. The method of claim 9 wherein the density of CD3+ cells and the density of CD8+ cells is determined in the tumor biopsy sample.

11. The method of claim 7 wherein the one or more immune markers comprise the expression level of one or more genes from the group consisting of CCR2, CD3D, CD3E, CD3G, CD8A, CXCL10, CXCL11, GZMA, GZMB, GZMK, GZMM, IL15, IRF1, PRF1, STAT1, CD69, ICOS, CXCR3, STAT4, CCL2, and TBX21.

12. The method of claim 7 wherein the one or more immune markers comprise the expression level of one or more genes from the group consisting of GZMH, IFNG, CXCL13, GNLY, LAG3, ITGAE, CCL5, CXCL9, PF4, IL17A, TSLP, REN, IHH, PROM1 and VEGFA.

13. The method according of claim 7 wherein the one or more immune markers comprise an expression level of at least one gene representative of human adaptive immune response and an expression level of at least one gene representative of human immunosuppressive response.

14. The method of claim 13 wherein the at least one gene representative of human adaptive immune response is selected from the group consisting of CCL5, CCR2, CD247, CD3E, CD3G, CD8A, CX3CL1, CXCL11, GZMA, GZMB, GZMH, GZMK, IFNG, IL15, IRF1, ITGAE, PRF1, STAT1 and TBX21 and said at least one gene representative of human immunosuppressive response is selected from the group consisting of CD274, CTLA4, IHH, IL17A, PDCD1, PF4, PROM1, REN, TIM-3, TSLP, and VEGFA.

15. The method of claim 7 wherein the immune response is assessed by a scoring system that involves the steps of:
a) quantifying the one or more immune markers in a tumor biopsy sample obtained from said patient;
b) comparing each values-obtained at step a) for said one or more immune markers with a distribution of values obtained for each of said one or more immune markers from a reference group of patients suffering from said cancer;
c) determining for each values obtained at step a) for said one or more immune markers the percentile of the distribution to which the values obtained at step a) correspond;
d) calculating the arithmetic mean value or the median value of percentile.

16. The method of claim 15 wherein the immune response is assessed by a continuous-scoring system comprising the steps of:
a) quantifying the density of CD3+ cells and the density of CD8+ cells in a tumor biopsy sample obtained from said patient;
b) comparing each density values obtained at step a) with a distribution of values obtained from a reference group of patients suffering from said cancer;
c) determining for each density values obtained at step a) the percentile of the distribution to which the values obtained at step a) correspond;
d) calculating the arithmetic mean value of percentile.

17. The method of claim 15 wherein the immune response is assessed by a non-continuous scoring system that involves the steps of:
a) quantifying the density of CD3+ cells and the density of CD8+ cells in a tumor biopsy sample obtained from said patient;
b) comparing each density values obtained at step a) with a distribution of values obtained from a reference group of patients suffering from said cancer;
c) determining for each density values obtained at step a) the percentile of the distribution to which the values obtained at step a) correspond;
d) calculating the arithmetic mean value of percentile; and
e) comparing the arithmetic mean value obtained at step d) with a predetermined reference arithmetic mean value of percentile, and
f) assigning a low or high score depending on whether the arithmetic mean value of percentile is respectively lower or higher than the predetermined reference arithmetic mean value of percentile.

18. The method of claim 15 wherein the immune response is assessed by a non-continuous scoring system that involves the steps of:
a) quantifying the density of CD3+ cells and the density of CD8+ cells in a tumor biopsy sample obtained from said patient;
b) comparing each density values obtained at step a) with a distribution of values obtained from a reference group of patients suffering from said cancer;
c) determining for each density values obtained at step a) the percentile of the distribution to which the values obtained at step a) correspond;
d) calculating the arithmetic mean value of percentile; and
e) comparing the arithmetic mean value of percentile obtained at step d) with 2 predetermined reference arithmetic mean values percentile, and
f) assigning a low, intermediate or high score when the arithmetic mean value:
is lower than the lowest predetermined reference arithmetic mean value of percentile
is comprised between the 2 predetermined reference arithmetic mean values of percentile or
is higher than the highest predetermined reference arithmetic mean value of percentile, respectively.

19. The method of claim 1 wherein the clinical response is determined by the assessment of the level of ctDNA.

20. The method of claim 1 wherein the clinical response is determined by the assessment of reduction in tumor volume that is assessed by imaging.

21. The method of claim 20 wherein the clinical response is assessed by radiography, ultrasound imaging, magnetic resonance, scintigraphy, or Tomography Emission Positron/Computed Tomography (PET/CT).

22. The method of claim 1 wherein the clinical response is assessed by the ycTNM scoring system.

23. The method of claim 1 that comprises the steps of:
a) assessing at least two parameters, wherein the first parameter is the immune response determined before the preoperative adjuvant therapy and the second parameter is the clinical response determined after the preoperative adjuvant therapy
b) implementing an algorithm on data comprising the parameters assessed at step a) as to obtain an algorithm output, the implementing step being computer-implemented; and
c) determining the risk of recurrence and/or death from the algorithm output obtained at step b).

24. The method of claim 1, wherein when the clinical response is ycTNM=0-I, the higher the arithmetic mean or median value of percentile, the lower is the risk of recurrence and/or death, the longer is the survival time of the patient, and an organ preservation strategy is implemented.

25. The method of claim 1, wherein when the clinical response is ycTNM=0-I, and the arithmetic mean or median value of percentile is classified as “high”, the patient has a low risk of recurrence and/or death, the survival time of the patient is long, and an organ preservation strategy is implemented.
